(19) United States
(12) Patent Application Publication
(10) Pub. No.: US 2023/0211023 A1
(43) Pub. Date: Jul. 6, 2023

(54) REPORTER SYSTEM FOR RADIONUCLIDE IMAGING

(71) Applicant: Erasmus University Medical Center Rotterdam, Rotterdam (NL)

(72) Inventor: Laura MEZZANOTTE, Rotterdam (NL)

(21) Appl. No.: 17/907,171

(22) PCT Filed: Mar. 25, 2021
(86) PCT No.: PCT/NL2021/050200

§ 371 Date: Sep. 23, 2022

(30) Foreign Application Priority Data
Mar. 25, 2020 (EP) 20165617.0

(51) Int. Cl. A61K 51/08; C12Q 1/66; C12N 15/86

(52) U.S. Cl. CPC A61K 51/088; C12N 15/86; C12Q 1/66

(57) ABSTRACT

The present invention provides a reporter system comprising (i) a gene expression construct for expression in a cell of a reporter gene, said reporter gene encoding a fusion protein comprising a transmembrane domain fused in-frame to a reporter domain, wherein said transmembrane domain upon insertion of the fusion protein into the cell membrane anchors the fusion protein in the cell membrane while expressing the reporter domain at the cell surface, and (ii) a reporter peptide labeled with a radiolabel, wherein said reporter domain comprises the large polypeptide subunit of a split luciferase, and wherein said reporter peptide comprises the small peptide subunit of said split luciferase, wherein both subunits associate by complementation to assemble into a luciferase complex.

### FIELD OF THE INVENTION

The present invention is in the field of medicine, in particular in gene therapy and cell therapy treatments. The present invention inter αliα provides products and methods for tracking of cells following their infusion, such as T-cells in chimeric antigen receptor (CAR) T-cell therapy. The present invention further provides products and methods for use in oncolytic virus therapy and other gene/cell therapies in general.

### BACKGROUND OF THE INVENTION

Pre-clinical and early clinical development of novel therapies, such as T-cell therapy or oncolytic viral therapy, greatly benefit from reliable tracking methodologies for cells or viruses after their infusion in subjects for therapeutic purpose. Imaging of molecular and cellular therapies is critical to understanding variability in treatment response, effectiveness of new treatment strategies, and for patient safety monitoring. In vivo imaging has shown to possess some unique features making it an ideal approach for the tracking of primary immune responses to cancer in experimental systems and translation of results from small animals to patients. In vivo imaging is non-invasive, yields whole body information, provides kinetic information by dynamic imaging, and enables standardization.

One successful way of tracking comprises immuno positron emission tomography (PET), also referred to as mAb imaging, wherein cells are monitored using monoclonal antibodies targeted to specific cell surface markers or receptors and labeled with radionuclides. Immuno-PET can for instance be used to allow the in vivo visualization of CD8-positive tumor infiltrating lymphocytes (TILs) in patients. In vivo imaging studies have shown that such CD8+-TILs have predictive value for T-cell treatments in a preclinical solid tumor model.

Another method for tracking may make use of reporter genes, either endogenous or heterologously-expressed. The human sodium iodide symporter (hNIS) (2.2Kb) can be used as a reporter gene in clinical monitoring of CAR T-cell therapy, whereby symporter activity in the transduced T-cells is visualized as intracellular accumulation of the technetium-99m pertechnetate (^{99m}TcO4-) probe by SPECT imaging. PET imaging is also supported in such methods when using an ¹²⁴I probe.

Although advantageous for its low immunogenicity, the use of the NIS reporter gene system has its limitations. For instance, it is naturally expressed in thyroid, stomach, salivary glands, mammary glands, and sometimes breast cells. Moreover tracer probes are not trapped and can efflux, which results in short imaging windows. Most importantly, the NIS reporter gene is a relatively long which hampers easy cloning into therapeutic cells or virus.

Hence, there is an unmet need for a more specific PET/SPECT reporter which is small and easy to clone and can be expressed in cells.

### SUMMARY OF THE INVENTION

The present invention provides products and methods for nuclear imaging based on reporter gene expression, wherein the reporter gene specifically binds the nuclear probe with high affinity, with the advantage that the reporter gene is short and can be easily cloned into therapeutic viral vectors used in gene therapy or into cells used in cell therapy in order to monitor these therapies using PET/SPECT.

The products and methods described herein provide for a system of indirect imaging wherein cellular and molecular processes are examined and linked to the expression of the reporter gene. In comparison to currently available PET/SPECT reporter genes and other indirect methods of labelling cells, the present invention is unique and highly specific.

One unique benefit of the present invention is that the reporter gene supports bioluminescence (BL) imaging in addition to PET/SPECT imaging. The hybrid BL/PET/SPECT reporter gene may accordingly find application in methods of cell tracking in diagnostic/prognostic settings, or in therapeutic methods, both as a (companion) diagnostic, or as a therapeutic.

The present invention now provides in a first aspect a reporter system comprising:
a gene expression construct for expression in a cell of a reporter gene, said reporter gene encoding a fusion protein comprising a transmembrane domain fused in-frame to a reporter domain, wherein said transmembrane domain upon insertion of the fusion protein into the cell membrane anchors the fusion protein in the cell membrane while expressing the reporter domain at the cell surface;
a reporter peptide labeled with a radiolabel;
wherein said reporter domain comprises, preferably consists of, the large polypeptide subunit of a split luciferase, and wherein said reporter peptide comprises, preferably consists of, the small peptide subunit of said split luciferase, wherein both subunits associate by complementation to assemble into a (preferably luminescent) luciferase complex.

In a preferred embodiment of the system of the present invention, said reporter domain consists of the large polypeptide subunit of a split luciferase, and wherein said reporter peptide consists of the small peptide subunit of said split luciferase.

In another preferred embodiment of the system of the present invention, the small peptide subunit has high affinity for the large polypeptide subunit of said split luciferase. As used herein, the term “high affinity” describes an intermolecular interaction between two entities that is of sufficient strength to produce detectable complex formation under physiologic or assay conditions. The term “high affinity” as used herein means that the two subunits associated with Kd less than 0.1 µM, more preferably less than 10 nM, still more preferably less than 1 nM, still more preferably between 0.1 and 1 nM, still more preferably between 0.5 and 1 nM.

In another preferred embodiment of the system of the present invention, the split luciferase may be selected from firefly (Photinus pyralis) luciferase (FLuc), click beetle (e.g. Pyrophorus plagiophthalamus) luciferase, Gaussia (e.g. Gaussia princeps) luciferase (GLuc), Renilla (e.g. Renilla reniformis) luciferase (RLuc), Oplophorus (e.g. Oplophorus gracilirostris) luciferase (OLuc; NanoLuc), and bacterial luciferase (Lux). Most preferably, the split luciferase is preferably NanoLuc.

In yet another preferred embodiment of the system of the present invention, the reporter peptide has a length of 9-30 amino acid residues. Preferably, the length of the reporter peptide is between 10-25 amino acids. Such as 11-22 amino acids. Preferably, the reporter peptide is not cleaved in blood.

In yet another preferred embodiment of the system of the present invention, the large polypeptide subunit comprises the amino acid sequence of SEQ ID NO:48 or an amino acid sequence having a sequence identity of at least 90%, preferably at least 95%, to SEQ ID NO:48, said sequence identity being determined over the entire length of the amino acid sequence, and wherein said amino acid sequence having a sequence identity of at least 90%, preferably at least 95%, to SEQ ID NO:48 binds the reporter peptide, preferably at high binding affinity with a dissociation constant Kd of less than 0.1 µM, more preferably less than 10 nM, still more preferably less than 1 nM, still more preferably between 0.1 and 1 nM, still more preferably between 0.5 and 1 nM.

In yet another preferred embodiment of the system of the present invention, the small peptide subunit comprises, preferably consists of, the amino acid sequence of any of SEQ ID NOs:28-46.

In still another preferred embodiment of the system of the present invention, the reporter domain is fused at its C-terminal end to said transmembrane domain.

In still another preferred embodiment of the system of the present invention, the transmembrane domain is selected from the transmembrane domain of proteins PDGFR, CD8, B7 protein, TLR4, CD4, neurexin3b, Notch receptor polypeptide, CD28, CD137 (41BB), CD3C and other truncated human type I and II transmembrane proteins, optionally in combination with a cytoplasmic domain of said protein (which may serve to enhance surface expression), preferably wherein said transmembrane domain comprises a sequence selected from the group consisting of SEQ ID NOs:1-11.

In still another preferred embodiment of the system of the present invention, the fusion protein further comprises a leader peptide fused in-frame to the transmembrane domain, preferably at the N-terminus. The leader sequence preferably comprises or is a signal peptide (which may serve to target the fusion protein to the cellular membrane). The leader sequence in the reporter gene is preferably positioned directly upstream (5’) from the initiation codon of the transmembrane domain sequence, or may contain the initiation codon. The leader sequence preferably encodes a signal peptide (which may serve to target the fusion protein to the cellular membrane). The leader sequence is preferably selected from the group consisting of, but not limited to, the leader sequence of human or mouse IgK, CD8, OSM, IgG2 H, BM40, Secrecon, IgKVIII, CD33, tPA, chymotrypsinogen, trypsinogen-2, IL-2, albumin (HSA), and insulin, preferably wherein said leader sequence comprises a sequence selected from the group consisting of SEQ ID NOs:12-26.

In still another preferred embodiment of the system of the present invention, the gene expression construct further comprises regulatory elements, such as promoters or poly A sequences.

In still another preferred embodiment of the system of the present invention, the reporter peptide comprises a radiolabel, which is preferably coupled to the reporter peptide through a chelator. Preferably, the chelator is coupled to the reporter peptide through a linker.

In preferred embodiments of the system of the present invention, the radiolabel is selected from ⁵¹Cr, ⁵²Fe, ^{52m}Mn, ⁶²Cu, ⁶⁴Cu, ⁶⁷Cu, ⁶⁷Ga, ⁶⁸Ga, ⁷²As, ⁷⁷As, ⁸⁹Zr, ⁹⁰Y, ⁹⁷Ru, ⁹⁹Tc (e.g. ^{99m}Tc), ¹⁰⁵Rh, ¹⁰⁹Pd, ¹¹¹In, ¹¹¹Ag, ^{113m}In, ¹²¹Sn, ¹²⁴I, ¹²⁷Te, ¹⁴²Pr, Wr, ¹⁴⁹Pm, ¹⁵¹Pm, ¹⁵³Sm, ¹⁵⁷Gd, ¹⁵⁹Gd, ¹⁶¹Tb, ¹⁶⁵Dy, ¹⁶⁶Ho, ¹⁶⁹Er, ¹⁶⁹Yb, ¹⁷²Tm, ¹⁷⁵Yb, ¹⁷⁷Lu, ¹⁸⁶Re, ¹⁸⁸Re, ¹⁹⁸Au, ¹⁹⁹Au, ²⁰³Pb, ¹¹C, ¹⁸F, ¹⁵O, and ¹³N.

In a highly preferred embodiment of the system of the present invention, the radiolabel is ¹¹¹In.

In another highly preferred embodiment of the system of the present invention,, the chelator is 1,4,7, 10-tetraazacyclododecane-N, N, N″, N‴-tetraacetic acid (DOTA).

In a highly preferred embodiment of the system of the present invention, the linker is 6-aminohexanoic acid (6ahx).

In preferred embodiments of the system of the present invention, the linker is linked to the reporter peptide through a Valine residue.

In preferred embodiments of the system of the present invention, the fusion protein is engineered to display the reporter domain on the surface of cells.

In preferred embodiments of the system of the present invention, the gene expression construct is comprised in a vector. The vector preferably comprising a promoter operably linked to a transcriptional unit encoding a reporter gene as described herein, preferably wherein the reporter gene is operably linked to a eukaryotic signal sequence.

In further preferred embodiments of the system of the present invention, the vector is comprised in a recombinant cell, preferably a T-cell.

In alternative preferred embodiments of the system of the present invention, the vector is comprised in a viral genome, including the genome of an oncolytic virus such as an adenovirus, reovirus, measles virus, herpes simplex virus, Newcastle disease virus, vaccinia virus, senecavirus, enterovirus RIGVIR, semliki forest virus, vesicular stomatitis virus, and poliovirus, or the genome of a virus for cell transformation, such as a retrovirus or lentivirus.

In another aspect, the present invention provides a reporter peptide that comprises a small peptide subunit of a split luciferase, which small peptide subunit associates by complementation to the large polypeptide subunit of said split luciferase to assemble into a luciferase complex, wherein the small peptide subunit has high affinity for the large polypeptide subunit, wherein the reporter peptide has a length of 9-30 amino acid residues, and wherein said reporter peptide is labeled with a radionuclide, preferably suitable for use in PET or SPECT, and wherein said radionuclide is preferably coupled to said reporter peptide through a chelator and linker.

The split luciferase is preferably selected from firefly (Photinus pyralis) luciferase (FLuc), click beetle (e.g. Pyrophorus plagiophthalamus) luciferase, Gaussia (e.g. Gaussiaprinceps) luciferase (GLuc), Renilla (e.g. Renilla reniformis) luciferase (RLuc), Oplophorus (e.g. Oplophorus gracilirostris) luciferase (OLuc; NanoLuc), and bacterial luciferase (Lux), preferably wherein the split luciferase is NanoLuc.

In preferred embodiments of the reporter peptide of the present invention, the length of the reporter peptide is between 10-25 amino acids. Such as 11-22 amino acids. Preferably, the reporter peptide is not cleaved in blood.

In preferred embodiments of the reporter peptide of the present invention, the radiolabel is selected from ⁵¹Cr, ⁵²Fe, ^{52m}Mn, ⁶²Cu, ⁶⁴Cu, ⁶⁷Cu, ⁶⁷Ga, ⁶⁸Ga, ⁷²As, ⁷⁷As, ⁸⁹Zr, ⁹⁰Y, ⁹⁷Ru, ⁹⁹Tc (e.g. ^{99m}Tc), ¹⁰⁵Rh, ¹⁰⁹Pd, ¹¹¹In ¹¹¹Ag, ^{113m}In, ¹²¹Sn, ¹²⁴I, ¹²⁷Te, ¹⁴²Pr, ¹⁴³Pr, ¹⁴⁹Pm, ¹⁵¹Pm, ¹⁵³Sm, ¹⁵⁷Gd, ¹⁵⁹Gd, ¹⁶¹Tb, ¹⁶⁵Dy, ¹⁶⁶Ho, ¹⁶⁹Er, ¹⁶⁹Yb, ¹⁷²Tm, ¹⁷⁵Yb, ¹⁷⁷Lu, ¹⁸⁶Re, ¹⁸⁸Re, ¹⁹⁸Au, ¹⁹⁹Au, ²⁰³Pb, ¹¹C, ¹⁸F, ¹⁵O, and ¹³N. Preferably, the radiolabel is ¹¹¹In.

In preferred embodiments of the reporter peptide of the present invention, the chelator is 1,4,7,10-tetraazacyclododecane-N, N, N″, N‴-tetraacetic acid (DOTA).

In preferred embodiments of the reporter peptide of the present invention, the linker is 6-aminohexanoic acid (6ahx).

In preferred embodiments of the reporter peptide of the present invention, the reporter peptide comprises, preferably consists of, the sequence of any one of SEQ ID NOs:28-46.

In another aspect, the present invention provides a pharmaceutical composition for infusion into the body of a subject comprising the reporter peptide of the present invention.

In another aspect, the present invention provides a pharmaceutical composition for infusion into the body of a subject comprising the vector or recombinant cell comprising the gene expression construct of the reporter system of the present invention.

In another aspect, the present invention provides a pharmaceutical combination for simultaneous, separate or sequential infusion into the body of a subject comprising:
a pharmaceutical composition for infusion into the body of a subject comprising the vector or recombinant cell comprising the gene expression construct of the reporter system of the present invention, and
a pharmaceutical composition comprising the reporter peptide of the present invention.

In another aspect, the present invention provides a

The term “pharmaceutical composition” in aspects of this invention indicated herein above may take the form of a “diagnostic composition”, indicating that the purpose of the composition is diagnostic, rather than therapeutic.

The present invention also provides a method for treating disease or monitoring of disease treatment comprising administering to a subject in need thereof a therapeutically or diagnostically effective amount of the reporter system according to the present invention, or the pharmaceutical combination of the present invention.

Possible diseases treated or monitored by the use of the reporter system of this invention may include cancer therapy using oncolytic viruses or (CAR) T-cells.

In another aspect, the present invention provides a cell, preferably a human or animal cell, more preferably a non-human mammalian cell, transduced with the gene expression construct of the of the reporter system according to the present invention.

### DESCRIPTION OF THE DRAWINGS

FIG. 1 shows the light signals collected after the reaction of different concentration of HiBiT peptide with TMLgBiT expressed in the membrane of HEK-293 cells.

FIG. 2 shows the calculation of Kd of the reaction between LgBiT protein and HiBiT peptide using one site specific binding function. The calculated Kd for HiBiT is 6.8 nM. The calculated Kd for Hibit-DOTA is 1,3 nM. The calculated Kd for Hibit-6ahx-DOTA is 0.7 nM.

FIG. 3 shows the CPM from gamma counter of cells expressing TMLgBiT and control cells after addition of 1 nmol of radioactive [¹¹¹In]-DOTA-6ahx-HiBiT peptide.

FIG. 4 shows a SPECT image of a life mouse injected with cells expressing the TMLgBiT reporter (right flank) and not expressing the TMLgBiT reporter (left flank) after infusion of the radiolabeled HiBiT peptide. High specific signal is detected.

FIG. 5 shows the nucleic acid sequence of the pBiT1.1-C [TK/LgBiT] Vector of Example 1. Vector sequence reference points: Base pairs: 3865; HSV-TK promoter: 27-779; MCS: 815-865; LgBiT: 903-1376; SV40 late poly(A) signal 1410-1631; ColE1-derived plasmid replication origin: 1956-1992; Beta-lactamase (Ampr) coding region: 2747-3607 (Reverse).

FIG. 6 shows the biodistribution of the radiolabeled HiBiT-6ahx-Dota peptide 1h after administration as described in Example 1, showing that it is prevalently and rapidly eliminated by kidneys and that there is no a-specific accumulation in other tissues.

FIG. 7 shows a SPECT images of life mice bearing PC3-TMLgBiT tumors that were injected with DOTA-6ahx-HiBiT (left) and [¹¹¹In]-DOTA-6ahx-HiBiT (Right). BL image taken 30 min after injection of 1 nM DOTA-6ahx-HiBiT and intraperitoneal injection of NanoLuc substrate (left). Dynamic SPECT scan performed for 1 hour after injection of 0,13 nM of [¹¹¹In]DOTA-6ahx-HiBiT (Right). In both cases specific signal from tumor is visible.

FIG. 8 shows the biodistribution of [¹¹¹In]-DOTA-6ahx-HiBiT and of 100-fold excess of DOTA-6ahx-HiBiT (in case of blocking study) in mice bearing PC3-TMLgBiT tumors. Effective blocking of signal in organs is achieved.

### DETAILED DESCRIPTION OF THE INVENTION

The term “reporter gene”, as used herein, refers to a gene encoding a reporter protein the expression of which is readily quantifiable or observable through the use of a “probe”. Because gene regulation usually occurs at the level of transcription, transcriptional regulation and promoter activity are often assayed by quantitation of gene products. In aspects of this invention the reporter gene, inter αliα, encodes a protein domain displayed at the cell surface (reporter domain) that is recognized by an externally provided probe composed of a labeled peptide (reporter peptide) that associates with the reporter domain of the reporter protein through protein-fragment complementation of high affinity.

Protein-fragment complementation assays, such as the NanoLuc luciferase complementation assay is commonly used to infer protein-protein interaction based on weak association between the LgBiT polypeptide fragment and the SmBiT peptide fragment. The basis of this assay lies in split complementation assays, wherein the bioluminescent enzyme is split into two separate components, which reassemble and form a functional bioluminescent complex. The strength of these assays lies not only in the surprisingly strong tendency of bioluminescent proteins to form their native and active structures, but also in the assays’ applicability in imaging and in vivo studies.

In aspects of the present invention the reporter gene encodes a reporter protein that represents the large fragment of a split bioluminescent protein (for instance the large fragment of NanoLuc, LgBiT) that is expressed and anchored in the membrane of cells through a transmembrane domain fused to the reporter protein. The probe used for detection of the reporter protein is formed by a small peptide that represents the small complementary fragment of a split bioluminescent protein (for instance the HiBiT peptide of NanoBiT) which has a high specific affinity for the (mostly N-terminal) large fragment of the split bioluminescent protein, meaning that the two fragments have nanomolar affinity for each other. The reporter gene is expressed in cells (e.g. in in vitro transduced T-cells), which cells can then be traced in vivo in a subject by PET/SPECT imaging after addition of the probe to the subjects circulation.

As an experimental demonstration, the present inventors show that in vitro transfected HEK-293 cells expressing the reporter gene TMLgBit (TM for transmembrane) when subcutaneously injected into a mouse, could be detected in vivo by the injection of 111In -DOTA-6ax-HiBit peptide through PET/SPECT imaging after 1 hour.

The new reporter system allows for the diagnostic and prognostic imaging of cell therapies (especially T cell therapies).

The luminescent protein NanoLuc® (Nluc), as discovered and further developed by Hall et al. (ACS Chem. Biol. 2012, 7, 1848-1857), was engineered by directed evolution from a deep-sea shrimp ( Oplophorus gracilirostris) luciferase. The enzyme’s luminescence was optimized with the identification of a novel substrate obtained by synthesis and screening of coelenterazine analogs. The Nluc protein is a 19.1 kDa, monomeric, highly soluble and stable, ATP-independent enzyme. The optimal substrate, furimazine, produces a glow type luminescence (half-life > 2 h) with a higher specific activity than that of Firefly luciferase (Fluc) or Renilla luciferase (Rluc). The high luminescence intensity of Nluc, its high solubility and its small size compared to Fluc (61 kDa), or to Rluc (36 kDa) make that Nluc is considered a potent tool for in vitro protein-protein interaction assays.

In traditional luciferase complementation assays (LCAs) for the detection of protein-protein interactions (PPIs) within living cells using bioluminescence, complementary DNA (cDNA) of luciferase is first split into the N- and C- terminal fragments and then fused to cDNAs of a protein pair of interest. A cell of interest is transformed or transfected with the resulting recombinant cDNAs so that a pair of the recombinant proteins is expressed within the cell. When the recombinant proteins interact with each other, the enzymatic activity of the split luciferase is reconstituted. Compared with other assays that detect protein-protein interaction in living cells, these assays have a high dynamic range of interaction signals due to extremely low background signals in the samples.

One such a luciferase complementation assay based on Nluc was described by Dixon et al. (ACS Chem. Biol. 2016. Vol 11:400-408). This system, referred to as NanoLuc® Binary Technology (NanoBiT), is a two-subunit system based on NanoLuc® luciferase that can be applied to the intracellular detection of PPIs. Large BiT (LgBiT; 18 kDa, SEQ ID NO:47 and 48) and Small BiT (SmBiT; 11 amino acid peptide) subunits are expressed as fusions to proteins of interest, where PPI facilitates subunit complementation to give a bright, luminescent enzyme. Unlike related approaches where an enzyme or protein is simply split, LgBiT was independently optimized for structural stability and SmBiT was selected from a peptide library specifically for the PPI application. The result is a subunit pair that weakly associates (Kd = 190 pM) yet still maintains 30% of the activity of full-length NanoLuc at saturation. In contrast to many split systems, the LgBiT:SmBiT interaction is reversible, allowing the detection of rapidly dissociating proteins. A NanoBiT-reporter assay, was recently developed by Botta et al. (J Biol Chem. 2019. Vol 294(45):16587-16603). The technique is essentially used to study PPI in membrane protein complexes.

The term “split luciferase” is used herein in its art-recognized manner to refer to a luciferase protein that is split into an N- and C-terminal domain, both of which, when taken alone, are non-functional in that they do not emit luminescence, whereas when the two non-functional halves are brought into close enough proximity, complementation or reconstitution of the domains restores luciferase activity. The functional enzyme exhibits emission of light upon the addition of an appropriate substrate such as Furimazine. Split luciferase is a well-known term in the context of protein-fragment complementation assay (PCA) technology, in particular bimolecular fluorescence complementation assays for the identification and quantification of protein-protein interactions (PPI), such as the Split Luciferase Complementation Assay (SLCA) (Paulmurugan et al., Proc Natl Acad Sci USA. 2002;99:15608-15613; Deng et αl., J Virol Methods. 2011 Sep; 176(1-2): 108-111).

Although the low affinity of this split luciferase complementation assay (the SmBiT small peptide has (Kd > 100 µM) to LgBiT) may be beneficial for PPI testing, the present invention is based on the use of a small peptide with a high affinity to the large polypeptide fragment of the split luciferase. This high-affinity peptide is herein referred to as the reporter peptide, and may elsewhere be referred to as complementation reporter. In the instance of the HiBiT peptide (VSGWRLFKKIS [w/o Met] or MVSGWRLFKKIS [w/Met]; Dixon et al., ACS Chem. Biol. 2016, 11, 400-408; WO2016/040835) (1.3-kDa), its Kd is 0.7 nM for binding to LgBiT.

Other peptides that have shown to have high affinity to the NLuc include NVSGWRLFKKISN (NLpep78; Kd=3,4 nM, Dixon et al., 2016), NVTGYRLFKKISN (NLpep79; Kd=8,5 nM, Dixon et al., 2016), VSGWRLFKKISN (NLpep80, Dixon et al.,2016), and those indicated in W02014151736A1 (below) and W02016040835A1, such as NLpep83 (w/ Met) MNVSGWRLFKKIS. Binding affinities can be determined as disclosed inter αliα in WO2014151736A1 (e.g. Example 26), wherein a NanoGlo Luciferase Assay Reagent (Promega Corporation) or PBS+0.1% Prionex® protein stabilizer with Furimazine was added to the binding pairs whose affginity is to be determined (the non-luminescent polypeptide and the non-luminescent peptide), and shaken at room temperature for 10 minutes, whereafter luminescence was detected on a GloMax with 0.5 s integration, and Kd values were determined using Graphpad Prism, One Site-Specific Binding. The dissociation constants can be measured under various buffer conditions (e.g. PBS for complementation then NanoGlo buffer for detection; PBS for complementation and detection; or NanoGlo buffer for complementation and detection), preferably in PBS.

Table 1: Suitable reporter peptide sequences comprising concensus sequence VSGWRLFKKIS for use as probe (HiBiT) in conjunction with the LgBiT polypeptide reporter domain of the transmembrane fusion protein. The reporter peptides in aspects of this invention may or may not comprise a Met residue at the N-terminus (table below taken from W02014151736A1).

High affinity (spontaneous) peptides

| | | | | 157 | | 159 | | 161 | | 163 | | 165 | | 167 | | 169 |
|---|---|---|---|---|---|---|---|---|---|---|---|---|---|---|---|---|
| Wt | T | I | N | G | V | T | G | W | R | L | C | E | K | I | L | A |
| 78 | | | | N | V | S | G | W | R | L | F | K | K | I | S | N |
| 80 | | | | - | V | S | G | W | R | L | F | K | K | I | S | N |
| 80* | | | | - | V | S | G | W | R | L | F | K | K | 1 | S | A |
| 83 | | | | N | V | S | G | W | R | L | F | K | K | I | S | - |
| 83* | | | | G | V | S | G | W | R | L | F | K | K | I | S | - |
| 86 | | | | - | V | S | G | W | R | L | F | K | K | I | S | - |
| 80+S* | | | | S | V | S | G | W | R | L | F | K | K | I | S | A |
| 80-s | | | | S | V | S | G | W | R | L | F | K | K | I | S | N |
| 80+NS* | | | N | S | V | S | G | W | R | L | F | K | K | I | S | A |
| 80+NS | | | N | S | V | S | G | W | R | L | F | K | K | I | S | N |
| 83+S | | | S | N | V | S | G | W | R | L | F | K | K | I | S | - |
| 83+5* | | | S | G | V | S | G | W | R | L | F | K | K | I | S | - |
| 83+NS | | N | S | N | V | S | G | W | R | L | F | K | K | I | S | - |
| 83+NS* | | N | S | G | V | S | G | W | R | L | F | K | K | I | S | - |
| 86+S | | | | S | V | S | G | W | R | L | F | K | K | I | S | - |
| 86+NS | | | N | S | V | S | G | W | R | L | F | K | K | I | S | - |
| 78+S | | | S | N | V | S | G | W | R | L | F | K | K | I | S | N |
| 78+NS | | N | S | N | V | S | G | W | R | L | F | K | K | I | S | N |

A preferred reporter peptide in embodiments of this invention is the 11-amino acid HiBiT peptide having the sequence VSGWRLFKKIS (SEQ ID NO:25). HiBiT and LgBiT efficiently form a stable complex that acts as the active binding pair to detect radionuclides via in vivo imaging.

In highly preferred embodiments of aspects of this invention, use is made of beneficial aspects of the NanoBiT-reporter assay. The present invention, in some embodiments, may link a radioactive label to the HiBiT subunits and thereby provides for a method for immune cell tracking. This method is superior to tracking of luciferase activity, which is not easily measurable in vivo. Hence, the present inventors propose, in some embodiments of this invention, for the use of a radioactively labeled HiBiT peptide as they surprisingly found that HiBiT and LgBiT can be used in the reporter gene system proposed herein because the protein affinity between LgBiT and HiBiT is essentially maintained. An advantage is that the LgBit subunit is small (only 0.8 kb) and easy to clone, and in combination with a radioactively labeled HiBiT subunit, allows multimodal and highly specific imaging. It has now been shown that T cells expressing a transmembrane LgBit can be produced, and T cells can tracked in vivo by simple addition of radiolabeled SmBit.

The present invention thus provides for a method for diagnostic and prognostic imaging useful in, for instance, T-cell tracking in T-cell therapies. The tracking of cells or oncolytic viruses after infusion also finds application in animal studies, such as used in preclinical research.

The methods of the invention allow for non-invasive in vivo imaging and yield whole-body information. PET imaging may for instance be performed using a ¹²⁴I tracer, whereas single-photon emission computerized tomography (SPECT) may for instance be performed using ^{99m}TcO4. Use may concurrently be made of the bioluminescent aspects of the reconstituted and peptide reporter with the reporter domain of the fusion protein.

The presently proposed BL/PET/SPECT reporter gene is beneficially small, and is easy to clone, and allows for multimodal and highly specific imaging. The SPECT method can be used for diagnostic/prognostic imaging of cell therapies; especially T-cell therapies, oncololytic viruses and gene therapy in general (nanoparticles, etc.). The present inventors have found that the new reporter gene may acts as a specific reporter gene for SPECT/PET imaging when the high-affinity reporter peptide (optionally linked to a chelator) is used as the radiolabeled tracer. As described in one embodiment herein, the exemplary TMLgBit and DOTA-linker-HiBit peptide represent one embodiment of a new system for SPECT/PET imaging in vivo and represents a preferred embodiment in aspects of this invention.

As envisioned by the present inventors, alternative embodiments of the present invention may be based on different types of luciferase, commonly used to detect protein-protein interactions, including embodiments based on firefly (Photinus pyralis) luciferase known as FLuc, sea pansy (Renilla reniformis) luciferase known as RLuc, copepod (Gaussia princeps) luciferase known as GLuc, click beetles (P. plagiophthalamus and Cratomorphus distinctus) luciferase known as CBR and ELuc, respectively, or deep sea shrimp (Oplophorus gracilirostri) luciferase known as NanoLuc. or NLuc. The present invention is not based on the enzymatic character of the luciferases. Rather, the essential element in the present invention relates to the presence of a small peptide subunit of luciferase that complements the main subunit of the luciferase, as exemplified herein for the case of NanoLuc. One may for instance use luciferase complementation subunits from other luciferases comprising a large peptide expressed in a cell, and preferable expressed as a transmembrane, and comprising a small subunit peptide that can be radiolabeled. The small subunit peptide may be longer than 20 amino acids, such as 22-25 amino acids. Such combinations of alternatives to LgBit and HiBit as described herein are envisioned as embodiments of this invention, wherein preferably the radiolabeled reporter peptides are not longer than about 30 amino acids, and are preferably not cleaved in blood.

The reporter in this invention is expressed as a transmembrane fusion protein, wherein the reporter domain is the extracellular part and an arbitrary transmembrane domain is used to display the reporter domain on the outside of the cell. The transmembrane domain of the fusion protein allows for retention of the reporter domain at the cell surface. The term “transmembrane domain” as used herein, refers to any membrane-spanning protein domain as a region of the protein’s polypeptide chain that is self-stabilizing and that folds independently from the rest. “Transmembrane domain”, as the term is used herein, includes any part of the cell membrane-spanning protein. The transmembrane domain of proteins may share common structural features, for example, an α-helical stretch of 21-26 hydrophobic amino acids, such as isoleucine, valine, phenylalanine, tryptophan, methionine. The term “membrane-spanning” as used herein refers to a protein (also referred to herein as a polypeptide) which associates with the plasma membrane of a cell and extends from the intracellular or cytoplasmic domain to the extracellular or outer domain of the cell. The reporter domain is therefore preferably fused at its C-terminal end to a transmembrane domain.

The transmembrane domain can be derived either from a natural or from a synthetic source. Where the source is natural, the domain can be derived from any membrane-bound or transmembrane protein. Transmembrane regions of particular use for the purposes herein may be derived from (i.e., comprise at least the transmembrane region(s) of) a member selected from the group: the alpha, beta or zeta chain of the T-cell receptor; CD28, CD3 epsilon, CD45, CD4, CD5, CD8, CD9, CD16, CD22, CD33, CD37, CD64, CD80, CD86, CD 134, CD 137, and CD 154. Alternatively the transmembrane domain can be synthetic, in which case the transmembrane domain will comprise predominantly hydrophobic residues such as leucine and valine. In further embodiments, the transmembrane domain comprises the triplet of phenylalanine, tryptophan and valine at each end of a synthetic transmembrane domain.

In preferred embodiments of this invention, the transmembrane part of the fusion protein may for instance comprise the PDGFR transmembrane domain. In alternative preferred embodiments of this invention, the reporter domain may be fused to a CD8 transmembrane domain, e.g. corresponding to AA 183-203 of CD8, or to a CD4, neurexin3b Notch receptor polypeptide, CD28, CD137 (41BB), CD8a or CD3C transmembrane domain as the transmembrane domain of the fusion protein of the invention. Various other transmembrane carrier peptides or proteins may be used as transmembrane domain of the fusion protein for the extracellular expression of the reporter domain, as long as the reporter domain is allowed to interact outside the cell with the radiolabeled reporter peptide outside the cell.

In preferred embodiments, the fusion protein further comprises a leader sequence, preferably fused at the N-terminal end of the transmembrane domain in the fusion protein. Such a leader sequence may be selected from any suitable leader sequence effecting membrane directed expression of the fusion product. Suitable leader sequence may for instance include a mouse IgK chain leader sequence fused to the N-terminal end of the LgBIT peptide subunit. Alternatively, a human CD8 leader sequence may be employed, and is preferably similarly fused to the N-terminal end of the LgBIT peptide subunit. Further alternative leader sequences at the N-terminal end of the LgBIT peptide subunit may be selected from the group consisting of, but not limited to, leader sequences of Human OSM, Mouse Ig Kappa, Human IgG2 H, BM40, Secrecon, Human IgKVIII, CD33, tPA, Human Chymotrypsinogen, Human trypsinogen-2, Human IL-2, Albumin (HSA), and Human insulin.

The reporter peptide in aspects of this invention is preferably not expressed in cells. The reporter peptide in aspects of this invention is preferably administered in vivo to a subject provided with recombinant cells expressing the fusion protein as described herein.

The reporter peptide in aspects of this invention is preferably radiolabeled. The term radiolabel as used herein, includes reference to radionuclides. In certain aspects, the label may be a “radio-opaque” label, e.g., a label that can be easily visualized using x-rays. Radio-opaque materials are well known to those of skill in the art. The most common radiopaque materials include iodide, bromide or barium salts. Other radio-opaque materials are also known and include, but are not limited to organic bismuth derivatives, radiopaque polyurethanes, organobismuth composites, radiopaque barium polymer complexes, and the like. Preferred radiolabels, include for example radioactive labels and/or labels detected by MRI, NMR, PET, and the like. Highly preferred radiolabels include, but are not limited to, ⁵¹Cr, ⁵²Fe, ^{52m}Mn, ⁶²Cu, ⁶⁴Cu, ⁶⁷Cu, ⁶⁷Ga, ⁶⁸Ga, ⁷²As, ⁷⁷As, ⁸⁹Zr, ⁹⁰Y, ⁹⁷Ru, ⁹⁹Tc, ¹⁰⁵Rh, ¹⁰⁹Pd, ¹¹¹In, ¹¹¹Ag, ^{113m}In, ¹²¹Sn, ¹²⁷Te, ¹⁴²Pr, ¹⁴³Pr, ¹⁴⁹Pm, ¹⁵¹Pm, ¹⁵³Sm, ¹⁵⁷Gd, ¹⁵⁹Gd, ¹⁶¹Tb, ¹⁶⁵Dy, ¹⁶⁶Ho, ¹⁶⁹Er, ¹⁶⁹Yb, ¹⁷²Tm, ¹⁷⁵Yb, ¹⁷⁷Lu, ¹⁸⁶Re, ¹⁸⁸Re, ¹⁹⁸Au, ¹⁹⁹Au, and ²⁰³Pb. Particular useful PET labels include, but are not limited to ¹¹C, ¹⁸F, ¹⁵O, ¹³N, and the like. Common labels used in MRI include, but are not limited to gadolinium chelates and iron oxide nanoparticles or microparticles with various surface modifications. Gadolinium chelates, such as gadopentate dimeglumine, are the most widely used paramagnetic contrast material. Iron oxide particles are part of a class of superparamagnetic MRI contrast agents. These compounds typically consist of magnetite (iron oxide) cores are coated with dextran or siloxanes, encapsulated by a polymer, or further modified.

The radiolabel is preferably bonded to the reporter peptide through the use of a chelator. To this end, the reporter peptide in aspects of this invention is preferably conjugated (through formation of coordination bonds) to a chelator to allow coupling between a radiolabel to the reporter peptide. Chelating groups are well known to those of skill in the art. In certain aspects, chelating groups are derived from ethylene diamine tetra-acetic acid (EDTA), diethylene triamine penta-acetic acid (DTPA), cyclohexyl 1,2-diamine tetra-acetic acid (CDTA), ethyleneglycol-O,O′-bis(2-aminoethyl)-N,N,N′,N′-tetra-acetic acid (EGTA), N,N-bis(hydroxybenzyl)-ethylenediamine-N,N′-diacetic acid (HBED), triethylene tetramine hexa-acetic acid (TTHA), 1,4,7,10-tetraazacyclododecane-N,N’-,N″,N‴-tetra-acetic acid (DOTA), hydroxyethyldiamine triacetic acid (HEDTA), 1,4,8,11-tetra-azacyclotetradecane-N,N′,N″,N‴-tetra-acetic acid (TETA), substituted DTPA, substituted EDTA, and the like. Examples of certain preferred chelators include unsubstituted or, substituted 2-iminothiolanes and 2-iminothiacyclohexanes, in particular 2-imino-4-mercaptomethylthiolane. One chelating agent, 1,4,7,10-tetraazacyclododecane-N, N, N″, N‴-tetraacetic acid (DOTA), is particularly preferred because of its ability to chelate a number of diagnostically and therapeutically important metals, such as radionuclides and radiolabels. Highly preferred chelators include DOTA and its derivatives cb-do2a, tcmc, TETA, CB-TE2A, CB-TE1A1P, DIAMSAR, NOTA and its derivatives, NETA, NETA-monamide, TACN_TM, DOTAGA, NODAGA, DTPA, CHX-A-DTPA, TRAP, AAZTA, H2dedpa, h4octapa, h2decapa,H2azapa, HBED, SHBED, BPCA, CP256, DFO, PCTA, p-SCN-Bn-DFO296, p-SCN-Bn-H6phospa, HEHA, and PEPA.

The chelator, in turn, may be bonded directly to the reporter peptide, but is preferably bonded to the reporter peptide through the use of a linker. A “linker” or “linking agent” as used herein, is a molecule that is used to join two or more molecules, and may also be referred to as a spacer. In certain aspects the linker is typically capable of forming covalent bonds to both molecule(s). Linking agents are well known to those of skill in the art. In preferred aspects of this invention, linkers may include, but are not limited to, 6-aminohexanoic acid (6ahx), 4-aminobutyric acid (GABA), (2-aminoethoxy) acetic acid (AEA), PEG2 Spacer (8-amino-3,6-dioxaoctanoic acid), PEG3 Spacer (12-amino-4,7,10-trioxadodecanoic acid), PEG4 Spacer (15-amino-4,7,10,13-tetraoxapenta-decanoic acid), 5-aminovaleric acid (Ava), Beta-alanine, and Ttds (Trioxatridecan-succinamic acid). Very good results have been obtained with direct coupling or using 6ahx as a linker, as these provided for increased affinity between the (fusion) peptides. The linker is preferably conjugated to a Valine residue of the reporter peptide as this coupling least affects the interaction (reconstitution) between the reporter peptide and reporter domain of the fusion protein.

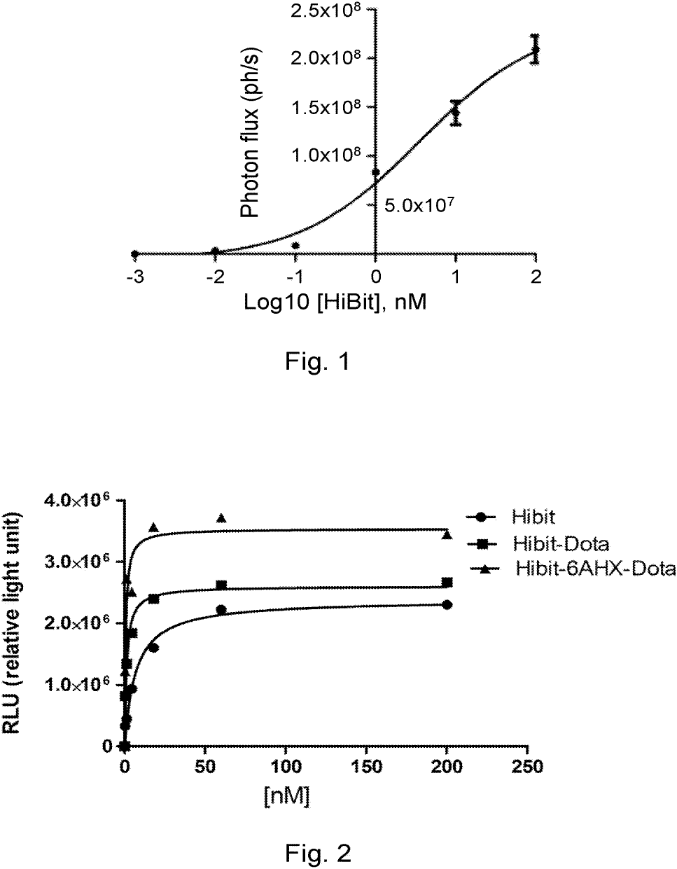

Alternatively, or in addition, the linker between the chelator and the reporter peptide reporter may comprise, or consist of, a non-covalent albumin-binding ligand. Such a linker may extend the circulating half-life of the radiolabeled reporter peptide. Non-limiting examples of such ligands are provided inter αliα in Zorzi et al. 2019 (Med. Chem. Commun. 10, 1068), in particular the Albumin-binding molecules in Tables 1-3, as well as the albumin-binding small organic compounds, albumin-binding peptide ligands, and albumin-binding protein ligands of FIGS. 2-4 therein, all of which are incorporated herein by reference.

The preferably chelated and radiolabeled reporter peptide is then preferably injected as a tracer in living subjects, and allowed to interact with cells expressing the fusion protein as described herein.

A highly preferred embodiment of a radiolabeled reporter peptide in aspects of this invention is [¹¹¹In]-DOTA-6ahx-HiBiT.

Proper reconstitution of the reporter domain of the fusion protein and the reporter peptide may be checked by in vitro testing of luminescence production using appropriate luciferin-based luminogenic substrates, d-luciferin (FLuc, CBR, and ELuc), coelenterazine or derivatives or analogs thereof, e.g., furimazine (RLuc, GLuc, and NanoLuc).

The present invention finds, inter alia, application in cell therapies (e.g. CAR or TCR T cells). Chimeric antigen receptor (CAR) T cells) are T cells that have been genetically engineered to produce an artificial T-cell receptor for use in immunotherapy. CARs are receptor proteins that have been engineered to give T cells the new ability to target a specific protein. The receptors are chimeric in that they combine both antigen-binding and T-cell activating functions into a single receptor. The premise of CAR-T immunotherapy is to modify T cells to recognize cancer cells in order to more effectively target and destroy them.

In CAR T cell therapy, T cells are harvest from patients, and transformed to express a specific CAR, which CAR programs the transformed T-cells to target an antigen that is present on the surface of tumors. CAR-T cells can be either derived from T cells in a patient’s own blood (autologous) or derived from the T cells of another healthy donor (allogeneic). The transformed T-cells are subsequently infused into patients to attack their tumors. After CAR-T cells are infused into a patient, they act as a “living drug” against cancer cells. When they come in contact with their targeted antigen on a cell, CAR-T cells bind to it and become activated, then proceed to proliferate and become cytotoxic. CAR-T cells destroy cells through several mechanisms, including extensive stimulated cell proliferation, increasing the degree to which they are toxic to other living cells (cytotoxicity) and by causing the increased secretion of factors that can affect other cells such as cytokines, interleukins and growth factors.

The present inventors now propose to include into these CAR T-cells, or in cells of other therapeutic cell therapies, a reporter gene of the present invention which enables radionuclide imaging of the therapeutic cell in vivo. The term “radionuclide imaging”, as used herein, refers to the non-invasive technique of inferring the distribution of radioactive tracers within (tissues of) the body of a subject by detecting the photons emitted due to decay of a tracer introduced into the body of a subject using (gamma) radiation detectors located outside of the subject under study.

One modality for radionuclide imaging foreseen by the present inventors comprises Positron Emission Tomography. The term “Positron Emission Tomography” or “PET”, as used herein, refers to one of two major nuclear imaging techniques currently in wide use, wherein the three-dimensional distribution of a tracer labelled with a positron emitter is measured. The acquisition is carried out by a set of detectors arranged around the subject. Positron emitters are radioactive isotopes (e.g. ¹¹C, ¹³N, ¹⁵O, ¹⁸F).

Another modality for radionuclide imaging foreseen by the present inventors comprises single photon emission computed tomography. The term “Single photon emission computed tomography” or “SPECT”, as used herein, refers to one of two major nuclear imaging techniques currently in wide use, wherein imaging is performed by using gamma rays to acquire projections from multiple angles. For cardiac purposes an arc of 180 degrees is preferred. Cross-sectional images are produced for all axial locations covered by the field of view (FOV) of the gamma camera, resulting in a stack of 2D images that form a 3D data set. The technique needs delivery of a gamma-emitting radioisotope (a radionuclide) into the patient, e.g. through injection into the bloodstream. In aspects of this invention, the radioisotope is attached to a small peptide to provide the tracer molecule. Radionuclides for use in SPECT include, for instance, ⁹⁹Y or ¹¹¹In. Other examples are provided herein below. The term “radionuclide imaging”, as used herein, includes reference to hybrid imaging systems (e.g. PET/CT, SPECT/CT and PET/MR).

Other details of the invention, include specific methods and technology for making and using the subject matter thereof, are described below.

### Expression Constructs and Transformation

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Maniatis, et al., Molecular Cloning, A Laboratory Manual (Cold Spring Harbor, 1990) and Ausubel, et al., 1994, Current Protocols In Molecular Biology (John Wiley & Sons, 1996), both incorporated herein by reference).

The term “expression vector” refers to any type of genetic construct comprising a nucleic acid coding for an RNA capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleotide sequences that serve other functions as well.

In certain embodiments, a plasmid vector is contemplated for use to in cloning and gene transfer. In general, plasmid vectors containing replicon and control sequences which are derived from species compatible with the host cell are used in connection with these hosts. The vector ordinarily carries a replication site, as well as marking sequences which are capable of providing phenotypic selection in transformed cells. In a non-limiting example, E. coli is often transformed using derivatives of pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, for example, promoters which can be used by the microbial organism for expression of its own proteins.

In addition, phage vectors containing replicon and control sequences that are compatible with the host microorganism can be used as transforming vectors in connection with these hosts. For example, the phage lambda GEM™-1 may be utilized in making a recombinant phage vector which can be used to transform host cells, such as, for example, E. coli LE392.

Bacterial host cells, for example, E. coli, comprising the expression vector, are grown in any of a number of suitable media, for example, LB. The expression of the recombinant protein in certain vectors may be induced, as would be understood by those of skill in the art, by contacting a host cell with an agent specific for certain promoters, e.g., by adding IPTG to the media or by switching incubation to a higher temperature. After culturing the bacteria for a further period, generally of between 2 and 24 h, the cells are collected by centrifugation and washed to remove residual media.

Many prokaryotic vectors can also be used to transform eukaryotic host cells. However, it may be desirable to select vectors that have been modified for the specific purpose of expressing proteins in eukaryotic host cells. Expression systems have been designed for regulated and/or high level expression in such cells. For example, the insect cell/baculovirus system can produce a high level of protein expression of a heterologous nucleic acid segment, such as described in U.S. Pat. Nos. 5,871,986 and 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®.

Other examples of expression systems include STRATAGENE®’S COMPLETE CONTROL™ Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

### Regulatory Signals

The construct may contain additional 5’ and/or 3’ elements, such as promoters, poly A sequences, and so forth. The elements may be derived from the host cell, i.e., homologous to the host, or they may be derived from distinct source, i.e., heterologous.

A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30-110 bp upstream of the start site, although a number of promoters have been shown to contain functional elements downstream of the start site as well. To bring a coding sequence “under the control of” a promoter, one positions the 5’ end of the transcription initiation site of the transcriptional reading frame “downstream” of (i.e., 3’ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid molecule, as may be obtained by isolating the 5’ non-coding sequences located upstream of the coding segment and/or exon. Such a promoter can be referred to as “endogenous.” Similarly, an enhancer may be one naturally associated with a nucleic acid molecule, located either downstream or upstream of that sequence. Alternatively, certain advantages will be gained by positioning the coding nucleic acid segment under the control of a recombinant or heterologous promoter, which refers to a promoter that is not normally associated with a nucleic acid molecule in its natural environment. A recombinant or heterologous enhancer refers also to an enhancer not normally associated with a nucleic acid molecule in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the β-lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook, et al., 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally any promoter/enhancer combination (as per, for example, the Eukaryotic Promoter Data Base EPDB) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5’ methylated Cap dependent translation and begin translation at internal sites (Pelletier and Sonenberg, Nature, 334:320-325 (1988)). IRES elements from two members of the picornavirus family (polio and encephalomyocarditis) have been described (Pelletier and Sonenberg, supra), as well an IRES from a mammalian message (Macejak and Sarnow, Nature, 353:90-94 (1991))1991). All are useful in aspects of this invention. IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference). Stoichiometry of co-expression of multiple factors or multi-units of complex proteins or of multiple genes cloned in a single vector (i.e. in polycistronic vectors) may be improved by the use of 2A “self-cleaving” peptides, which are 18-22 amino-acid-long viral oligopeptides that mediate “cleavage” of polypeptides during translation in eukaryotic cells. Suitable examples for use in aspects of this invention include 2A sequences / peptides from e.g. foot-and-mouth disease virus (F2A), equine rhinitis A virus (E2A), porcine teschovirus-1 (P2A) or Thosea asigna virus (T2A) optionaly with a GSG linker at the N-terminus, the coding sequences for which may be inserted, in-frame, between two coding DNA sequences.

Suitable promoters for use in aspects of this invention include, but are not limited to, the CMV promoter, EF1-alpha, PGK1,SV40, CAGGS, UBC, human B-actin constitutive promoters, tissue specific promoters (e.g. CD2, CD8, CD3, TCF-1, promoters) and inducible promoters (e.g. NFAT, NFkb, TOX, TOX2, BATF3) and chemical inducible promoters (e.g. tetracycline or doxycycline-controlled transcriptional activation [Tet-On/Tet-Off] systems).

### Other Vector Sequence Elements

Vectors can include a multiple cloning site (MCS), which is a nucleic acid region that contains multiple restriction enzyme sites, any of which can be used in conjunction with standard recombinant technology to digest the vector (see, for example, Carbonelli, et al., FEMS Microbiol. Lett., 172(1):75-82 (1999), Levenson, et al., Hum. Gene Ther. 9(8): 1233-1236 (1998), and Cocea, Biotechniques, 23(5):814-816 (1997)), incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see, for example, Chandler, et al., 1997, herein incorporated by reference).

The vectors or constructs of the present invention will generally comprise at least one termination signal. A “termination signal” or “terminator” comprises a DNA sequence involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 adenosine residues (polyA) to the 3’ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and to minimize read through from the cassette into other sequences.

Terminators contemplated for use in the invention include any known terminator of transcription described herein or known to one of ordinary skill in the art, including but not being limited to, for example, the termination sequences of genes, such as the bovine growth hormone terminator, viral termination sequences, such as the SV40 terminator. In certain embodiments, the termination signal may be a lack of transcribable or translatable sequence, such as an untranslatable/untranscribable sequence due to a sequence truncation.

In expression, particularly eukaryotic expression, one will typically include a polyadenylation signal to effect proper polyadenylation of the transcript. The nature of the polyadenylation signal is not believed to be crucial to the successful practice of the invention, and any such sequence may be employed. Preferred embodiments include the SV40 polyadenylation signal or the bovine growth hormone polyadenylation signal, both of which are convenient, readily available, and known to function well in various target cells. Polyadenylation may increase the stability of the transcript or may facilitate cytoplasmic transport.

In order to propagate a vector in a host cell, it may contain one or more origins of replication sites which are specific nucleotide sequences at which replication is initiated. Alternatively, an autonomously replicating sequence (ARS) can be employed if the host cell is yeast.

### Transformation Methodology

Suitable methods for nucleic acid delivery for use with the current invention are believed to include virtually any method by which a nucleic acid molecule (e.g., DNA) can be introduced into a cell as described herein or as would be known to one of ordinary skill in the art. Such methods include, but are not limited to, direct delivery of DNA such as by ex vivo transfection (Wilson, et al., Science, 244:1344-1346 (1989), Nabel et al, Science, 244:1342-1344 (1989), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harlan and Weintraub, J. Cell Biol., 101(3):1094-1099 (1985); U.S. Pat. No. 5,789,215, incorporated herein by reference); by electroporation (U.S. Pat. No. 5,384,253, incorporated herein by reference; Tur-Kaspa, et al., Mol. Cell Biol., 6:716-718 (1986); Potter, et al., Proc. Natl. Acad. Sci. USA, 81:7161-7165 (1984); by calcium phosphate precipitation (Graham and Van Der Eb, Virology, 52:456-467 (1973); Chen and Okayama, Mol. Cell Biol., 7(8):2745-2752 (1987); Rippe, et al., Mol. Cell Biol., 10:689-695 (1990); by using DEAE-dextran followed by polyethylene glycol (Gopal, Mol. Cell Biol., 5:1188-190 (1985); by direct sonic loading (Fechheimer, et al., Proc. Natl. Acad. Sci. USA, 89(17):8463-8467 (1987); by liposome mediated transfection (Nicolau and Sene, Biochem. & Biophys. Acta., 721:185-190 (1982); Fraley, et al., Proc. Natl. Acad. Sci. USA, 76:3348-3352 (1979); Nicolau, et al., Meth. Enzym., 149:157-176 (1987); Wong, et al., Gene, 10:879-894 (1980); Kaneda, et al., Science, 243:375-378 (1989); Kato, et al., J. Biol. Chem., 266:3361-3364 (1991) and receptor-mediated transfection (Wu and Wu, J. Biol. Chem., 262:4429-4432 (1987); Wu and Wu, 1988); by PEG-mediated transformation of protoplasts (Omirulleh, et al., Plant Mol. Biol., 21(3):415-428 (1987); U.S. Pat. Nos. 4,684,611 and 4,952,500, each incorporated herein by reference); by desiccation/inhibition-mediated DNA uptake (Potrykus, et al. Mol. Gen. Genet., 199(2):169-177 (1985), and any combination of such methods.

Transduction of T-cells may for instance occur using viral or non-viral methods for gene transfer. Viral systems include for instance lentiviral or retroviral gene transfer methods. The gene expression construct for expression in a cell of a reporter gene may be expressed in a cell such as a T cell or a natural killer (NK) cell. The gene expression construct for expression in a cell of a reporter gene may be expressed as an integrating nucleic acid (e.g., a DNA integrated into the host genome using a transposase/transposon) or as a non- integrating nucleic acid (e.g., a mRNA delivered via a viral vector such as a lentivirus or retrovirus). The T cell or NK cell expressing the gene expression construct for expression in a cell of a reporter gene may then be administered in a pharmaceutical preparation or excipient to a subject such as a human patient to treat or prevent a disease (e.g., a cancer, a fungal infection, a bacterial infection, or a viral infection). In some embodiments, naked DNA or a suitable vector encoding the gene expression construct for expression in a cell of a reporter gene can be introduced into a subject’s T cells (e.g., T cells obtained from a human patient with cancer or other disease). Methods of stably transfecting T cells by electroporation using naked DNA are known in the art. See, e.g., U.S. Pat. No. 6,410,319. Naked DNA generally refers to the DNA encoding a gene expression construct for expression in a cell of a reporter gene of the present invention contained in a plasmid expression vector in proper orientation for expression. In some embodiments, the use of naked DNA may reduce the time required to produce T cells expressing the gene expression construct for expression in a cell of a reporter gene generated via methods of the present invention. Alternatively, a viral vector (e.g., a retroviral vector, adenoviral vector, adeno-associated viral vector, or lentiviral vector) can be used to introduce the gene expression construct into T cells. Generally, a vector encoding a gene expression construct that is used for transfecting a T cell from a subject should be non-replicating in the subject’s T cells. A large number of vectors are known that are based on viruses, where the copy number of the virus maintained in the cell is low enough to maintain viability of the cell. Illustrative vectors include the pFB-neo vectors (STRATAGENE®) as well as vectors based on HIV, SV40, EBV, HSV, or BPV. Alternatively, transposon systems, such as Sleeping Beauty or PiggyBack, constitute non-viral methods for gene transfer and present a cost-efficient alternative to the expensive production of good manufacturing practice (GMP)-compliant virus for clinical application. One skilled in the art will readily know how to apply DNA plasmid systems, including the transposon systems composed of a transposase and a transposon of Sleeping Beauty or PiggyBack, now regularly used for therapeutic human cell genetic engineering.

Once it is established that the transfected or transduced T cell is capable of expressing the gene expression construct as a surface membrane protein with the desired regulation and at a desired level, it can be determined whether the reporter is functional in the host cell to provide for the desired binding to the complementary radionuclide-labelled HiBit peptide. Subsequently, the transduced T cells may be reintroduced or administered to the subject to activate anti-tumor responses in the subject. To facilitate administration, the transduced T cells may be made into a pharmaceutical composition or made into an implant appropriate for administration in vivo, with appropriate carriers or diluents, which are preferably pharmaceutically acceptable. The means of making such a composition or an implant have been described in the art (see, for instance, Remington’s Pharmaceutical Sciences, 16th Ed., Mack, ed. (1980)). Where appropriate, transduced T cells expressing a reporter gene can be formulated into a preparation in semisolid or liquid form, such as a capsule, solution, injection, inhalant, or aerosol, in the usual ways for their respective route of administration. Means known in the art can be utilized to prevent or minimize release and absorption of the composition until it reaches the target tissue or organ, or to ensure timed-release of the composition. Generally, a pharmaceutically acceptable form is preferably employed that does not ineffectuate the cells expressing the reporter gene. Thus, desirably the transduced T cells can be made into a pharmaceutical composition containing a balanced salt solution such as Hanks’ balanced salt solution, or normal saline.

### EXAMPLES

### Example 1

### Chimeric Transmembrane LgBiT

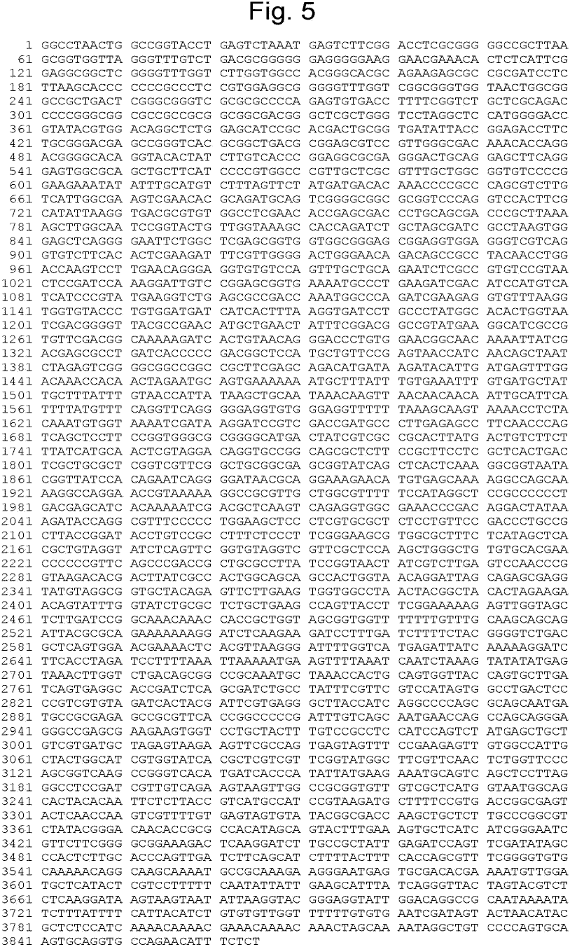

The transmembrane LgBiT sequence was created by inserting the LgBiT sequence that was cut from pBiT1.1-C [TK/LgBiT] (See FIG. 5; Promega Corporation, Madison, WI, USA) with restriction enzymes BglII and SalI into the cloning site of the pDisplay™ vector (Thermos Fisher Scientific, Waltham, Ma, USA), a mammalian expression vector for cell-surface protein display wherein the recombinant protein is fused at its N-terminus to the murine Ig _{K}-chain leader sequence for secretory pathway processing, and wherein the recombinant protein is fused at its C-terminus to the platelet derived growth factor receptor (PDGFR) transmembrane domain for anchoring the protein to the plasma membrane and allowing extracellular display. In this manner, a construct comprising a coding sequence for a chimeric, or fusion, protein is created comprising (from 5’-3’ in the nucleic acid and from N to C terminus in the expressed fusion protein) IgG _{K}-chain leader sequence - LgBiT sequence - PDGFR transmembrane domain sequence. The full sequence for the chimeric protein was then cut from the pDisplay™ vector using BamHI and NotI and inserted into the multiple cloning site of a pCDH-EF1-MCS lentiviral vector (System Biosciences, Palo Alto, CA, USA)to create the plasmid pCDH-EF1-LgBit, wherein the chimeric protein is under the control of the EF1-a promoter. Lentivirus particles were generated by means of transfection of HEK293 cells with packaging plasmids and the plasmid pCDH-EF1-LgBiT. Virus was quantified by antigen-capture ELISA, measuring HIV p24 levels (ZeptoMetrix Corporation, NY, USA). For transduction, PC3 cells (a human prostate cancer cell line) were resuspended in medium. Pseudoviral particles containing the chimeric transmembrane LgBiT construct were added to the cells (using 40 ng virus per 1 _{X} 10⁵ cells). Transduced PC3 cells were selected via serial dilution methods.

### Synthesis of HiBiT

The HiBiT peptide VSGWRLFKKIS was synthesized using a Nα-Fmoc solid-phase peptide synthesis strategy. The conjugation of Fmoc protected sequence (Val-Ser(tBu)-Gly-Trp(Boc)-Arg(Pbf)-Leu-Phe-Lys(Boc)-Lys(Boc)-Ile-Ser(tBu)) to the 2-chlorotrityl chloride resin was carried out in dimethylformamide (DMF) using hexafluorophosphate azabenzotriazole tetramethyl uronium (HATU) (3.8 equiv.) and N,N-diisopropylethylamine (DIPEA) (7.8 equiv.) for 45 minutes. Fmoc deprotection was accomplished by treatment of the resin with a 20% solution of piperidine in DMF. Amide formation and Fmoc deprotection were monitored by Kaiser test. Double couplings or Fmoc deprotection were performed when the reaction was not completed. The peptide synthesis was started by loading Fmoc-L-Ser(tBu)-OH (1.6 mmol, 4 equiv.) onto the solid support (0.25 g, loading capacity: 1.6 mmol/g). The resin was shaken for 90 min at room temperature. The resin was capped using dichloromethane/methanol/N,N-diisopropylethylamine (DCM/MeOH/DIPEA) (10 mL, 80:15:5, v/v/v) for 15 min at rt. Subsequent Fmoc deprotection and coupling with Fmoc-L-Ile-OH, Fmoc-L-Lys(Boc)-OH, Fmoc-L-Lys(Boc)-OH, Fmoc-L-Phe-OH, Fmoc-L-Leu-OH, Fmoc-L-Arg(Pbf)-OH, Fmoc-L-Trp(Boc)-OH, Fmoc-Gly-OH, Fmoc-L-Ser(tBu)-OH and Fmoc-L-Val-OH were achieved with 4 equivalent of the respective protected amino acids following the protocol described above.

### Synthesis of DOTA-6ahx-HiBiT

Conjugation of the linker to the N-terminal valine residue was accomplished by using Fmoc-6ahx-OH (2 equiv.), HATU (3.8 equiv.) and DIPEA (7.8 equiv.) in DMF. The resin was stirred for 2 h at rt. Then, the resin was washed trice with DMF and the Fmoc protecting group was removed by treatment of the resin with a 20% solution of piperidine in DMF. DOTA-tris(tBu) ester (3 equiv.) was coupled to the peptide in presence of benzotriazol-1-yl-oxytripyrrolidinophosphonium hexafluorophosphate (PyBoP) (3 equiv.), DIPEA (6 equiv.) and DMF. The reaction was carried out overnight at rt. Cleavage of the peptide from the solid support and its concomitant global deprotection were performed by treatment of the resin with a solution of trifluoroacetic acid/water/triisopropylsilane (TFA/H2O/TIPS) (95:2.5:2.5, v/v/v) for 6 h at rt. Upon filtration, the filtrate was collected, and the solvent was evaporated under vacuum. Trituration of the residue with cold diethyl ether gave the final crude product, which was purified by HPLC to give pure DOTA-6ahx-HiBiT, as a white solid (12 mg, 16.8%). ESI-MS: m/z 910.80 [M + 2H]2+, 922.21 [M + Na + H]2+ and 930.19 [M + 2Na]2+.

### Radiolabeling of DOTA-6ahx-HiBiT

¹¹¹InCl₃ (93.3 pL, 150 MBq) was added to a mixture of DOTA-6ahx-HiBiT (1 nmol), ascorbic acid/gentisic acid (10 pL, 50 mM), sodium acetate (1 pL, 2.5 M) and H2O (29.7 pL). The mixture was incubated for 20 min at 90° C. The reaction was monitored by instant thin-layer chromatography (iTLC) on silica gel impregnated glass fiber sheets eluted with a solution of sodium citrate (0.1 M, pH 5.0). The reaction mixture was cooled down for 5 min and diethylenetriaminepentaacetic acid (DTPA) (5 pL) was added to complex the remaining free indium-111. The radiochemical yield and molar activity of [¹¹¹In]-DOTA-6ahx-HiBiT were determined to be 93% and 150 MBq/nmol, respectively.

### Results

In the Example described above, an embodiment of new reporter gene for PET/SPECT imaging according to the invention is provided.

The reporter gene produced in this Example comprises a membrane expressed protein, based on the LgBiT part of NanoLuc while the tracer is a HiBiT peptide chemically modified with addition of a chelator and a linker.

First we assessed the functionality of the novel membrane-expressed LgBiT part. Cells expressing the transmembrane LgBiT (TMLgBiT) were seeded and HiBiT peptide was added together with Furimazine, the bioluminescent substrate. If the complementation between TMLgBiT and HiBiT peptide occurs, light is produced after addition of the substrate. FIG. 1 shows the light signals collected after the reaction of different concentration of HiBiT peptide with TMLgBiT expressed in the membrane of HEK-293 cells.

Then the HiBiT peptide was linked to the DOTA chelator using different types of linkers and affinity of the new DOTA-HiBiT peptide towards the LgBiT protein was evaluated in vitro.

The equilibrium dissociation constant (Kd) was determined as follow. An Opti-MEM solution (Thermo Fisher Scientific) with 10% Fetal Bovine Serum (FBS) was prepared where the LgBiT protein was diluted to a final concentration of 2 nM (each 500 µL) with a starting concentration of 200 nM in Opti-MEM plus 10% FBS. A three-time dilution series of synthetized peptides (DOTA-6ahx-HiBiT, DOTA-HiBiT) and native HiBiT was prepared and diluted: 150 µL of peptide solution with 350 µL of Opti-MEM plus 10% FBS. 90 µL of the above prepared solutions were added in triplicates to a white assay plate (Costar 3600) and 10 µL of 2 nM LgBiT solution (0.2 nM final concentration) was added to the wells with the peptide solutions and was incubated on an orbital shaker for 30 minutes at 600 RPM. A solution of furimazine (Promega) and 1 mM DTT (Thermo Fisher Scientific) was prepared in Opti-MEM plus 10% FBS and 10 µL was added to each well . The plate was incubated on orbital shaker at 600 RPM for 5 minutes, after addition of the solution. Luminescence was measured at the GloMax Multi Luminometer (Promega) with 0.5 s integration time per well. Kd was calculated using GraphPad Prism One site - specific binding.

The results showed that the affinity of the HiBiT peptide for LgBiT improves with about a 10-fold decrease in Kd, indicating that the reaction in vivo is not at all impaired. The HiBiT-6ahx-DOTA peptide (HiBiT-linker-DOTA) was selected for further testing due to increased stability. In FIG. 2, a calculation of the Kd of the reaction between LgBiT protein and HiBiT peptide is provided using one site specific binding function. The calculated Kd’s were 6,8 nM for HiBiT, 1,3 nM for HiBiT DOTA and 0,7 for Hibit-6ahx-DOTA.

Subsequently, the complementation reaction in live cells when the HiBiT-6ahx-DOTA is radiolabelled with ¹¹¹In (Indium) 150 mBq/nanomol for SPECT imaging was evaluated.

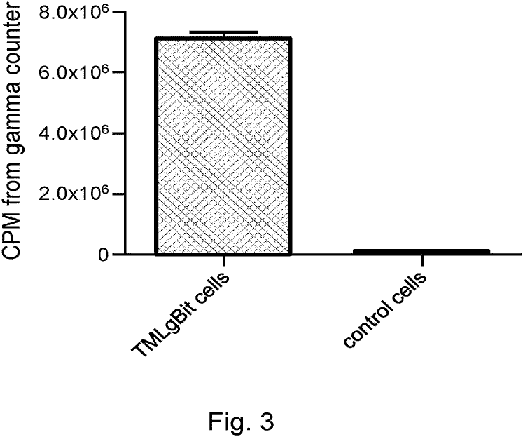

The [¹¹¹In]-DOTA-6ahx-HiBiT peptide (1 nM) is added to cells expressing the reporter gene TMLgBiT and cells not expressing TMLgBiT are used as negative control (20,000 cells/well). Cells are washed three times and radioactivity measured at a gamma counter. A clear difference in radioactive signal (2000 fold) resulted between TMLgBiT expressing cells and cells non expressing the reporter gene. In FIG. 3, the radiation (in CPM, measured using a gamma counter) of cells expressing TMLgBiT and control cells after addition of 1 nmol of radioactive [¹¹¹In]-DOTA-6ahx-HiBiT peptide is provided.

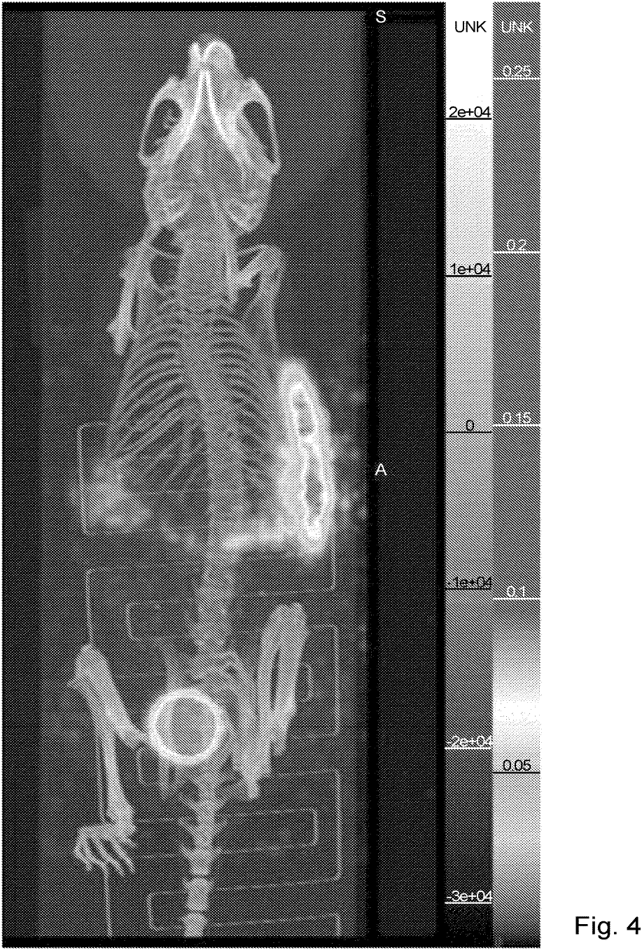

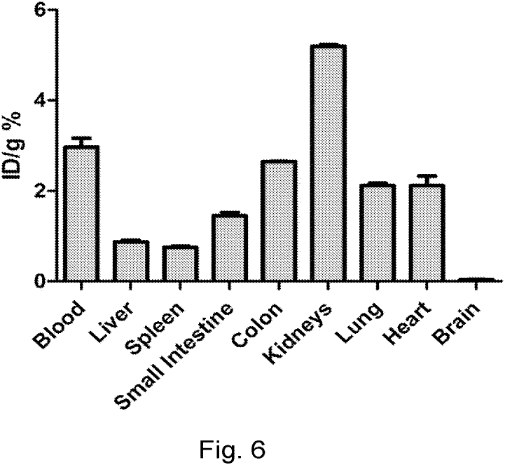

Finally in vivo experiments show that cells expressing the novel reporter gene TMLgBiT can be specifically detected using [¹¹¹In]-DOTA-6ahx-HiBiT peptide a dose of 20 mBq. FIG. 4 shows signals originated from subcutaneously injected TMLgBiT expressing cells 1 hour after injection of [¹¹¹In]-DOTA-6ahx-HiBiT peptide. Signal also originates from the bladder indicating the rapid elimination of the peptide through kidney and urine. Biodistribution data at 1 hour after injection were recorded and radioactivity concentrations in the various tissues was expressed as percentage of injected dose per gram tissue. As shown in FIG. 6, [¹¹¹In]-DOTA-6ahx-HiBiT is prevalently and rapidly eliminated by the kidneys (as all peptides) and there is no significant a-specific accumulation in other tissues.

In summary the data show that the new reporter gene system based on TMLgBiT protein acts as a specific reporter gene for SPECT/PET imaging when HiBiT peptide is linked to a chelator and radiolabelled. The combination of transmembrane (TM)LgBiT and DOTA-linker-HiBiT peptide represent a new system for SPECT/PET imaging in vivo. In FIG. 4, a SPECT image of a life mouse injected with cells expressing TMLgBiT (right flank) and not expressing the reporter (left flank) is provided. High specific signal is detected.

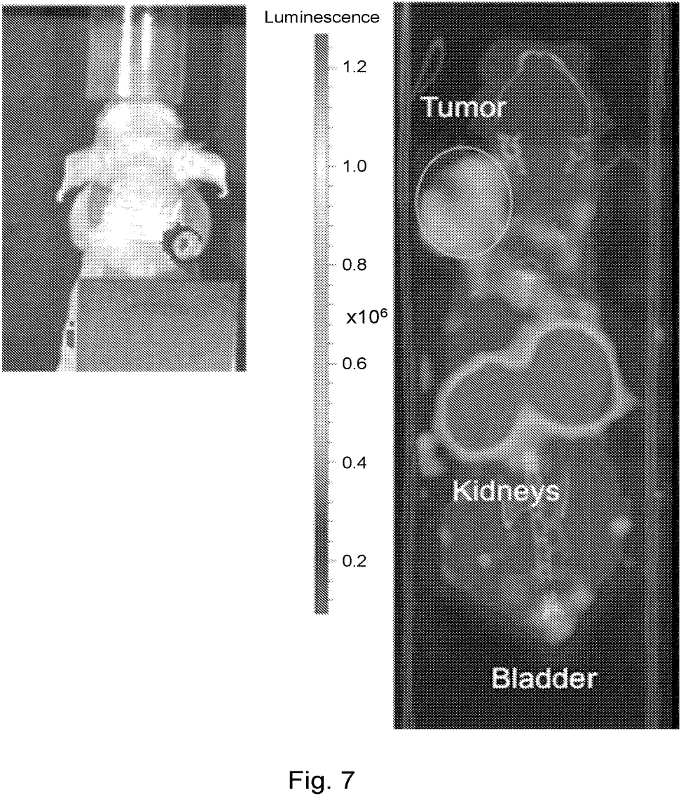

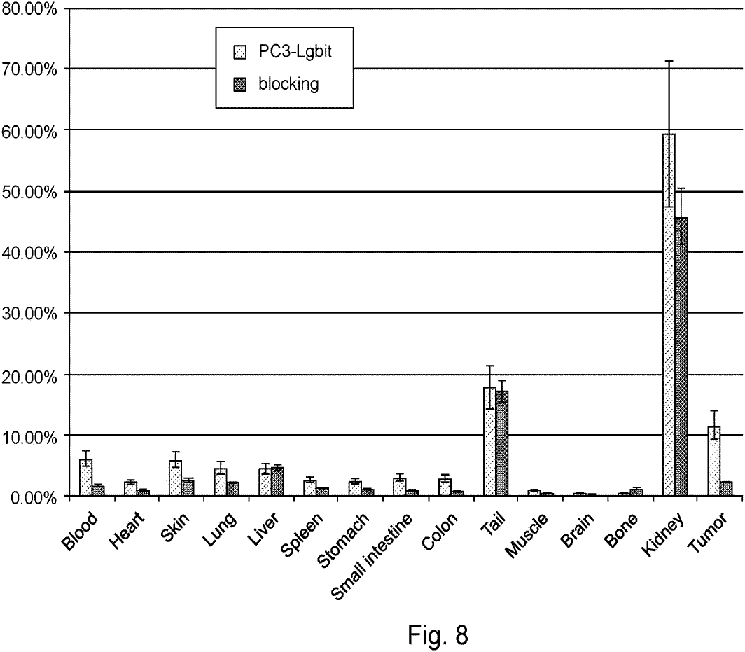

Additional experiments were performed and the results thereof are displayed in FIGS. 7 and 8. For these experiments, eight-week-old nude BALB/C male mice (n=12) were injected with 5x10⁶ PC-3-TM-LgBiT expressing cells (n=8). Cells were prepared for injections in PBS (Sigma-Aldrich) and matrigel (Corning) solution with a 50:50 ratio and a final injectable volume of 50 µL. Tumors were left to grow approximately 3-4 weeks post tumor cell implantation. To determine the functionality of the imaging system, a dynamic whole body SPECT/CT scan (VECT/CT Milabs) was performed. Mice were anesthetized using 1-2% isoflurane/O2 and the body temperature was maintained at 37° C. during the time of imaging (1 h) by using a heated bed aperture. The 1-hour dynamic SPECT/CT scan was performed immediately after tail vein injection of [¹¹¹In]-DOTA-6ahx-HiBiT for the PC-3 tumor model, (20 MBq labeled to 0.13 nmol in 200 µL PBS). In order to determine the inhibitory effect on PC-3 tumor uptake, 13 nmol of DOTA-6ahx-HiBiT (approximately 100-fold excess), was co-injected along with [¹¹¹In]-DOTA-6ahx-HiBiT (n=4). Dynamic scans were obtained over a total duration of 1 h with 30 time frames, directly after injection of [¹¹¹In]-DOTA-6ahx-HiBiT. Acquired images were reconstructed using SR-OSEM with 9 iterations and 128 subsets on a 36 × 36 × 35 mm matrix with 0.80 × 0.80 mm isotropic voxels. Images are displayed in FIG. 7.

Biodistribution was assessed by ex vivo analysis. For this, mice were sacrificed and dissected one hour after tail vein injection of approx. 20 MBq [¹¹¹In]-DOTA-6ahx-HiBiT peptide (n=4 per treated group/control). The organs (blood, heart, skin, lungs, liver, spleen, stomach, small intestine, colon, tail, muscle, brain, tumor, kidney and bone) were weighted and the radioactivity uptake in tumor and other organs was determined and expressed as percentage injected dose per gram of tissue (%ID/g). Tumors and organs were counted in a γ-counter (PerkinElmer). Counting time was 60 s per sample with an isotope-specific energy window and a counting error not exceeding 5%. After counting, the tumors were frozen in liquid nitrogen for further analysis.

TABLE 2

Sequences described herein

| Sequence | Type | Structural info | SEQ ID NO. |
|---|---|---|---|
| VVVISAILALVVLTIISLIILIML | aa | PDGFR transmembrane domain | 1 |
| IYIWAPLAGTCGVLLLSLVIT | aa | CD8 transmembrane domain | 2 |
| IHWWPISFIGVGLVLLIVLI | aa | B7 protein transmembrane domain | 3 |
| TIIGVSVLVVSVVAVLVYKFYF | aa | TRL4 transmembrane domain | 4 |
| MALIVLGGVAGLLLFIGLGIFF | aa | CD4 transmembrane domain | 5 |
| GMWGIVAAAALCILILLYAM | aa | neurexin3b transmembrane domain | 6 |
| FMYVAAAAFVLLFFVGCGVLL | aa | Notch receptor polypeptide transmembrane domain | 7 |
| WVLVVVGGVLACYSLLVTVAFII | aa | CD28 transmembrane domain | 8 |
| IISFFLALTSTALLFLLFFLTLRFSVV | aa | CD137 (41BB) transmembrane domain | 9 |
| IYIWAPLAGTCGVLLLSLVITLYCNHRN | aa | CD8a transmembrane domain | 10 |
| LCYLLDGILFIYGVILTALFL | aa | CD3C transmembrane domain | 11 |
| METDTLLLWVLLLWVPGST | aa | IgK chain leader sequence | 12 |
| MALPVTALLLPLALLLHAARP | aa | human CD8 leader sequence | 13 |
| MGVLLTQRTLLSLVLALLFPSMASM | aa | Human OSM leader sequence | 14 |
| METDTLLLWVLLLWVPGSTGD | aa | Mouse Ig Kappa leader sequence | 15 |
| MGWSCIILFLVATATGVHS | aa | Human IgG2 H leader sequence | 16 |
| MRAWIFFLLCLAGRALA | aa | BM40 leader sequence | 17 |
| MWWRLWWLLLLLLLLWPMVWA | aa | Secrecon leader sequence | 18 |
| MDMRVPAQLLGLLLLWLRGARC | aa | Human IgKVIII leader sequence | 19 |
| MPLLLLLPLLWAGALA | aa | CD33 leader sequence | 20 |
| MDAMKRGLCCVLLLCGAVFVSPS | aa | tPA leader sequence | 21 |
| MAFLWLLSCWALLGTTFG | aa | Human Chymotrypsinogen leader sequence | 22 |
| MNLLLILTFVAAAVA | aa | Human trypsinogen-2 leader sequence | 23 |
| MYRMQLLSCIALSLALVTNS | aa | Human IL-2 leader sequence | 24 |
| MKWVTFISLLFSSAYS | aa | Albumin (HSA) leader sequence | 25 |
| MALWMRLLPLLALLALWGPDPAAA | aa | Human insulin leader sequence | 26 |
| gtgagcggctggcggctgttcaagaagattagc | na | HiBiT | 27 |
| VSGWRLFKKIS | aa | HiBiT | 28 |
| MVSGWRLFKKIS | aa | HiBiT+M | 29 |
| NVSGWRLFKKISN | aa | NLpep78 | 30 |
| NVTGYRLFKKISN | aa | NLpep79 | 31 |
| VSGWRLFKKISN | aa | NLpep80 | 32 |
| MVSGWRLFKKISN | aa | NLpep80+M | 33 |
| MNVSGWRLFKKIS | aa | NLpep83+M | 34 |
| NVSGWRLFKKIS | aa | NLpep83 | 35 |
| GVSGWRLFKKIS | aa | NLpep83* | 36 |
| SVSGWRLFKKISA | aa | NLpep80+S* | 37 |
| SVSGWRLFKKISN | aa | NLpep80+S | 38 |
| NSVSGWRLFKKISA | aa | NLpep80+NS* | 39 |
| NSVSGWRLFKKISN | aa | NLpep80+NS | 40 |
| SNVSGWRLFKKIS | aa | NLpep83+S | 41 |
| SGVSGWRLFKKIS | aa | NLpep83+S* | 42 |
| SVSGWRLFKKIS | aa | NLpep86+S | 43 |
| NSVSGWRLFKKIS | aa | NLpep86+NS | 44 |
| SNVSGWRLFKKISN | aa | NLpep78+S | 45 |
| NSNVSGWRLFKKISN | aa | NLpep 78+NS | 46 |
| atggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagt | na | LgBiT | 47 |
| MVFTLEDFVGDWEQTAAYNLDQVLEQGGVSSLLQNLAVSVTPIQRIVRSGENALKIDIHVIIPYEGLSADQMAQIEEVFKWYPVDDHHFKVILPYGTLVIDGVTPNMLNYFGRPYEGIAVFDGKKITVTGTLWNGNKIIDERLITPDGSMLFRVTINS | aa | LgBiT | 48 |

1. A reporter system comprising:
(i) a gene expression construct for expression in a cell of a reporter gene, said reporter gene encoding a fusion protein comprising a transmembrane domain fused in-frame to a reporter domain, wherein said transmembrane domain upon insertion of the fusion protein into the cell membrane anchors the fusion protein in the cell membrane while expressing the reporter domain at the cell surface; and
(ii) a reporter peptide labeled with a radiolabel,
wherein said reporter domain comprises the large polypeptide subunit of a split luciferase, and wherein said reporter peptide comprises the small peptide subunit of said split luciferase, wherein both subunits associate by complementation to assemble into a luciferase complex.

2. The reporter system of claim 1, wherein said reporter domain consists of the large polypeptide subunit of a split luciferase, and wherein said reporter peptide consists of the small peptide subunit of said split luciferase, wherein the split luciferase is selected from firefly (Photinus pyralis) luciferase (FLuc), click beetle Pyrophorus plagiophthalamus) luciferase, Gaussia Gaussia princeps) luciferase (GLuc), Renilla (e.g. Renilla reniformis) luciferase (RLuc), Oplophorus Oplophorus gracilirostris) luciferase (OLuc; NanoLuc), and bacterial luciferase (Lux).

3. The reporter system of claim 1, wherein the small peptide subunit has high affinity for the large polypeptide subunit of said split luciferase, wherein the two subunits associated with Kd less than 0.1 µM, less than 10 nM, less than 1 nM, or between 0.5 and 1 nM.

4. The reporter system of claim 1, wherein the reporter peptide has a length of 9-30 amino acid residues, 10-25 amino acid residues, or 11-22 amino acid residues.

5. The reporter system of claim 1, wherein the large polypeptide subunit comprises the amino acid sequence of SEQ ID NO:48, or a sequence having a sequence identity of at least 90% to SEQ ID NO:48 and binding the small peptide subunit with Kd less than 0.1 µM, less than 10 nM, less than 1 nM, or between 0.5 and 1 nM.

6. The reporter system of claim 1, wherein the small peptide subunit has the amino acid sequence of any of SEQ ID NOs:28-46.

7. The reporter system of claim 1, wherein the transmembrane domain is selected from the transmembrane domain of proteins PDGFR, CD8, B7 protein, TLR4, CD4, neurexin3b, Notch receptor polypeptide, CD28, CD137 (41BB), CD3C and other truncated human type I and II transmembrane proteins, optionally in combination with a cytoplasmic domain of said protein, wherein said transmembrane domain comprises a sequence selected from the group consisting of SEQ ID NOs: 1-11.

8. The reporter system of claim 1, wherein the fusion protein further comprises a sequence of a leader peptide fused in-frame to the transmembrane domain, at the N-terminus, wherein the leader sequence is selected from the group consisting of a leader sequence of human or mouse IgK, CD8, OSM, IgG2 H, BM40, Secrecon, IgKVIII, CD33, tPA, chymotrypsinogen, trypsinogen-2, IL-2, albumin (HSA), and insulin, wherein said leader sequence comprises a sequence selected from the group consisting of SEQ ID NOs: 12-26.

9. The reporter system of claim 1, wherein the reporter peptide comprises a radiolabel, coupled to the reporter peptide through a chelator, wherein the chelator is coupled to the reporter peptide through a linker, wherein the radiolabel is selected from ⁵¹Cr, ⁵²Fe, ^{52m}Mn, ⁶²Cu, ⁶⁴Cu, ⁶⁷Cu, ⁶⁷Ga, ⁶⁸Ga, ⁷²As, ⁷⁷As, ⁸⁹Zr, ⁹⁰Y, ⁹⁷Ru, ⁹⁹Tc, ^{99m}Tc, ¹⁰⁵Rh, ¹⁰⁹Pd, ¹¹¹In, ¹¹¹Ag, ^{113m}In, ¹²¹Sn, ¹²⁴I, ¹²⁷Te, ¹⁴²Pr, ¹⁴³Pr, ¹⁴⁹Pm, ¹⁵¹Pm, ¹⁵³Sm, ¹⁵⁷Gd, ¹⁵⁹Gd, ¹⁶¹Tb, ¹⁶⁵Dy, ¹⁶⁶Ho, ¹⁶⁹Er, ¹⁶⁹Yb, ¹⁷²Tm, ¹⁷⁵Yb, ¹⁷⁷Lu, ¹⁸⁶Re, ¹⁸⁸Re, ¹⁹⁸Au, ¹⁹⁹Au, ²⁰³Pb, ¹¹C, ¹⁸F, ¹⁵O, and ¹³N.

10. The reporter system of claim 1, wherein the gene expression construct is comprised in a vector, wherein the vector is comprised in a recombinant cell, a T-cell; or wherein the vector is comprised in a viral genome, including the genome of an oncolytic virus including an adenovirus, reovirus, measles virus, herpes simplex virus, Newcastle disease virus, vaccinia virus, senecavirus, enterovirus RIGVIR, semliki forest virus, vesicular stomatitis virus, and poliovirus, or the genome of a virus for cell transformation, a retrovirus or lentivirus.

11. A reporter peptide comprising a small peptide subunit of a split luciferase, which small peptide subunit associates by complementation to the large polypeptide subunit of said split luciferase to assemble into a luciferase complex, wherein the small peptide subunit has high affinity for the large polypeptide subunit, wherein the reporter peptide has a length of 9-30 amino acid residues, and wherein said reporter peptide is labeled with a radionuclide, for use in PET or SPECT, and wherein said radionuclide is coupled to said reporter peptide through a chelator and linker.

12. The reporter peptide according to claim 11, wherein the split luciferase is selected from firefly (Photinus pyralis) luciferase (FLuc), click beetle Pyrophorus plagiophthalamus) luciferase, Gaussia Gaussia princeps) luciferase (GLuc), Renilla Renilla reniformis) luciferase (RLuc), Oplophorus Oplophorus gracilirostris) luciferase (OLuc; NanoLuc), and bacterial luciferase (Lux) .

13. A reporter peptide according to claim 11, wherein the reporter peptide has a length of 9-30 amino acid residues, 10-25 amino acid residues, or 11-22 amino acid residues, wherein the reporter peptide comprises the sequence of any one of SEQ ID NOs:28-46, and wherein the radiolabel is selected from ⁵¹Cr, ⁵²Fe, ^{52m}Mn, ⁶²Cu, ⁶⁴Cu , ⁶⁷C_{U}, ⁶⁷Ga, ⁶⁸Ga , ⁷²As, ⁷⁷As, ⁸⁹Zr, ⁹⁰Y, ⁹⁷Ru, ⁹⁹Tc, ¹⁰5Rh, ¹⁰⁹Pd, ¹¹¹In, ¹¹¹Ag, ^{113m}In, ¹²¹Sn, ¹²⁴I, ¹²⁷Te, ¹⁴²Pr, ¹⁴³Pr, ¹⁴⁹Pm, ¹⁵¹Pm, ¹⁵³Sm, ¹⁵⁷Gd, ¹⁵⁹Gd, ¹⁶¹Tb, ¹⁶⁵Dy, ¹⁶⁶Ho, ¹⁶⁹Er, ¹⁶⁹Yb, ¹⁷²Tm, ¹⁷⁵Yb, ¹⁷⁷Lu, ¹⁸⁶Re, ¹⁸⁸Re, ¹⁹⁸Au, ¹⁹⁹Au, ²⁰³Pb, ¹¹C, ¹⁸F, ¹⁵O, and ¹³N.

14. A pharmaceutical combination for simultaneous, separate or sequential infusion into the body of a subject comprising:
a) a pharmaceutical composition for infusion into the body of a subject comprising a vector or recombinant cell comprising the gene expression construct of the reporter system according to claim 1, and
b) a pharmaceutical composition comprising the reporter peptide comprising a small peptide subunit of a split luciferase, which small peptide subunit associates by complementation to the large polypeptide subunit of said split luciferase to assemble into a luciferase complex, wherein the small peptide subunit has high affinity for the large polypeptide subunit, wherein the reporter peptide has a length of 9-30 amino acid residues, and wherein said reporter peptide is labeled with a radionuclide for use in PET or SPECT, and wherein said radionuclide is coupled to said reporter peptide through a chelator and linker.

15. A method for treating disease or monitoring of disease treatment comprising administering to a subject in need thereof a therapeutically or diagnostically effective amount of the reporter system of claim 1, wherein said disease is cancer or wherein the disease treatment is cancer-therapy using oncolytic viruses or (CAR) T-cells.

16. A method for treating disease or monitoring of disease treatment comprising administering to a subject in need thereof a therapeutically or diagnostically effective amount of the pharmaceutical combination of claim 14, wherein said disease is cancer or wherein the disease treatment is cancer-therapy using oncolytic viruses or (CAR) T-cells.


SEQUENCE LISTING (54 sequences)

SEQ ID NO: 1 - protein, 24 aa, Homo sapiens
VVVISAILALVVLTIISLIILIML

SEQ ID NO: 2 - protein, 21 aa, Homo sapiens
IYIWAPLAGTCGVLLLSLVIT

SEQ ID NO: 3 - protein, 20 aa, Homo sapiens
IHWWPISFIGVGLVLLIVLI

SEQ ID NO: 4 - protein, 22 aa, Homo sapiens
TIIGVSVLVVSVVAVLVYKFYF

SEQ ID NO: 5 - protein, 22 aa, Homo sapiens
MALIVLGGVAGLLLFIGLGIFF

SEQ ID NO: 6 - protein, 21 aa, Homo sapiens
GMVVGIVAAAALCILILLYAM

SEQ ID NO: 7 - protein, 21 aa, Homo sapiens
FMYVAAAAFVLLFFVGCGVLL

SEQ ID NO: 8 - protein, 23 aa, Homo sapiens
WVLVVVGGVLACYSLLVTVAFII

SEQ ID NO: 9 - protein, 27 aa, Homo sapiens
IISFFLALTSTALLFLLFFLTLRFSVV

SEQ ID NO: 10 - protein, 28 aa, Homo sapiens
IYIWAPLAGTCGVLLLSLVITLYCNHRN

SEQ ID NO: 11 - protein, 21 aa, Homo sapiens
LCYLLDGILFIYGVILTALFL

SEQ ID NO: 12 - protein, 19 aa, Artificial Sequence
  IgK chain leader sequence
METDTLLLWVLLLWVPGST

SEQ ID NO: 13 - protein, 21 aa, Homo sapiens
MALPVTALLLPLALLLHAARP

SEQ ID NO: 14 - protein, 25 aa, Homo sapiens
MGVLLTQRTLLSLVLALLFPSMASM

SEQ ID NO: 15 - protein, 21 aa, Mus musculus
METDTLLLWVLLLWVPGSTGD

SEQ ID NO: 16 - protein, 19 aa, Homo sapiens
MGWSCIILFLVATATGVHS

SEQ ID NO: 17 - protein, 17 aa, Artificial Sequence
  BM40 leader sequence
MRAWIFFLLCLAGRALA

SEQ ID NO: 18 - protein, 21 aa, Artificial Sequence
  Secrecon leader sequence
MWWRLWWLLLLLLLLWPMVWA

SEQ ID NO: 19 - protein, 22 aa, Homo sapiens
MDMRVPAQLLGLLLLWLRGARC

SEQ ID NO: 20 - protein, 16 aa, Artificial Sequence
  CD33 leader sequence
MPLLLLLPLLWAGALA

SEQ ID NO: 21 - protein, 23 aa, Artificial Sequence
  tPA leader sequence
MDAMKRGLCCVLLLCGAVFVSPS

SEQ ID NO: 22 - protein, 18 aa, Homo sapiens
MAFLWLLSCWALLGTTFG

SEQ ID NO: 23 - protein, 15 aa, Homo sapiens
MNLLLILTFVAAAVA

SEQ ID NO: 24 - protein, 20 aa, Homo sapiens
MYRMQLLSCIALSLALVTNS

SEQ ID NO: 25 - protein, 16 aa, Artificial Sequence
  Albumin (HSA) leader sequence
MKWVTFISLLFSSAYS

SEQ ID NO: 26 - protein, 24 aa, Homo sapiens
MALWMRLLPLLALLALWGPDPAAA

SEQ ID NO: 27 - DNA, 33 bp, Artificial Sequence
  HiBiT
gtgagcggctggcggctgttcaagaagattagc

SEQ ID NO: 28 - protein, 11 aa, Artificial Sequence
  HiBiT
VSGWRLFKKIS

SEQ ID NO: 29 - protein, 12 aa, Artificial Sequence
  HiBiT + M
MVSGWRLFKKIS

SEQ ID NO: 30 - protein, 13 aa, Artificial Sequence
  NLpep78
NVSGWRLFKKISN

SEQ ID NO: 31 - protein, 13 aa, Artificial Sequence
  NLpep79
NVTGYRLFKKISN

SEQ ID NO: 32 - protein, 12 aa, Artificial Sequence
  NLpep80
VSGWRLFKKISN

SEQ ID NO: 33 - protein, 13 aa, Artificial Sequence
  NLpep80+M
MVSGWRLFKKISN

SEQ ID NO: 34 - protein, 13 aa, Artificial Sequence
  NLpep83+M
MNVSGWRLFKKIS

SEQ ID NO: 35 - protein, 12 aa, Artificial Sequence
  NLpep83
NVSGWRLFKKIS

SEQ ID NO: 36 - protein, 12 aa, Artificial Sequence
  NLpep83*
GVSGWRLFKKIS

SEQ ID NO: 37 - protein, 13 aa, Artificial Sequence
  NLpep80+S*
SVSGWRLFKKISA

SEQ ID NO: 38 - protein, 13 aa, Artificial Sequence
  NLpep80+S
SVSGWRLFKKISN

SEQ ID NO: 39 - protein, 14 aa, Artificial Sequence
  NLpep80+NS*
NSVSGWRLFKKISA

SEQ ID NO: 40 - protein, 14 aa, Artificial Sequence
  NLpep80+NS
NSVSGWRLFKKISN

SEQ ID NO: 41 - protein, 13 aa, Artificial Sequence
  NLpep83+S
SNVSGWRLFKKIS

SEQ ID NO: 42 - protein, 13 aa, Artificial Sequence
  NLpep83+S*
SGVSGWRLFKKIS

SEQ ID NO: 43 - protein, 12 aa, Artificial Sequence
  NLpep86+S
SVSGWRLFKKIS

SEQ ID NO: 44 - protein, 13 aa, Artificial Sequence
  NLpep86+NS
NSVSGWRLFKKIS

SEQ ID NO: 45 - protein, 14 aa, Artificial Sequence
  NLpep78+S
SNVSGWRLFKKISN

SEQ ID NO: 46 - protein, 15 aa, Artificial Sequence
  NLpep78+NS
NSNVSGWRLFKKISN

SEQ ID NO: 47 - DNA, 477 bp, Artificial Sequence
  LgBiT
atggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagt

SEQ ID NO: 48 - protein, 159 aa, Artificial Sequence
  LgBiT
MVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINS

SEQ ID NO: 49 - DNA, 3865 bp, Artificial Sequence
  vector
ggcctaactggccggtacctgagtctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaagtcgaacacgcagatgcagtcggggcggcgcggtcccaggtccacttcgcatattaaggtgacgcgtgtggcctcgaacaccgagcgaccctgcagcgacccgcttaaaagcttggcaatccggtactgttggtaaagccaccagatctgctagcgatcgcctaagtgggagctcaggggaattctggctcgagcggtggtggcgggagcggaggtggagggtcgtcaggtgtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagctaatctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagcggccgcaaatgctaaaccactgcagtggttaccagtgcttgatcagtgaggcaccgatctcagcgatctgcctatttcgttcgtccatagtggcctgactccccgtcgtgtagatcactacgattcgtgagggcttaccatcaggccccagcgcagcaatgatgccgcgagagccgcgttcaccggcccccgatttgtcagcaatgaaccagccagcagggagggccgagcgaagaagtggtcctgctactttgtccgcctccatccagtctatgagctgctgtcgtgatgctagagtaagaagttcgccagtgagtagtttccgaagagttgtggccattgctactggcatcgtggtatcacgctcgtcgttcggtatggcttcgttcaactctggttcccagcggtcaagccgggtcacatgatcacccatattatgaagaaatgcagtcagctccttagggcctccgatcgttgtcagaagtaagttggccgcggtgttgtcgctcatggtaatggcagcactacacaattctcttaccgtcatgccatccgtaagatgcttttccgtgaccggcgagtactcaaccaagtcgttttgtgagtagtgtatacggcgaccaagctgctcttgcccggcgtctatacgggacaacaccgcgccacatagcagtactttgaaagtgctcatcatcgggaatcgttcttcggggcggaaagactcaaggatcttgccgctattgagatccagttcgatatagcccactcttgcacccagttgatcttcagcatcttttactttcaccagcgtttcggggtgtgcaaaaacaggcaagcaaaatgccgcaaagaagggaatgagtgcgacacgaaaatgttggatgctcatactcgtcctttttcaatattattgaagcatttatcagggttactagtacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctct

SEQ ID NO: 50 - protein, 11 aa, Artificial Sequence
  Fmoc protected sequence
  MISC_FEATURE (2)..(2): Ser(tBu)
  MISC_FEATURE (4)..(4): Trp(Boc)
  MISC_FEATURE (5)..(5): Arg(Pbf)
  MISC_FEATURE (8)..(9): Lys(Boc)
  MISC_FEATURE (11)..(11): Ser(tBu)
VSGWRLFKKIS

SEQ ID NO: 51 - protein, 16 aa, Artificial Sequence
  High affinity peptide
TINGVTGWRLCERILA

SEQ ID NO: 52 - protein, 12 aa, Artificial Sequence
  NLpep80*
VSGWRLFKKISA

SEQ ID NO: 53 - protein, 14 aa, Artificial Sequence
  NLpep83+NS
NSNVSGWRLFKKIS

SEQ ID NO: 54 - protein, 14 aa, Artificial Sequence
  NLpep83+NS*
NSGVSGWRLFKKIS